(19) United States
(12) Patent Application Publication
(10) Pub. No.: US 2006/0160871 A1
(43) Pub. Date: Jul. 20, 2006

(54) Stable non-crystalline formulation comprising losartan

(75) Inventors: Srinivas Palakodaty, Foster City, CA (US); Jiang Zhang, Palo Alto, CA (US); Andreas Kordikowski, Bingley (GB); Linda S. Daintree, Todmorden (GB); Sarma Duddu, Redwood City, CA (US); Alan R. Kugler, Montara, CA (US); Herm Snyder, Pacifica, CA (US); David Lechuga-Ballesteros, San Jose, CA (US); Nagesh Palepu, Mill Creek, WA (US); Michael A. Eldon, Redwood City, CA (US)

Correspondence Address: Michael J. Mazza;Sedgwick Detert Moran & Arnold, LLP, One Market Plaza, Steuart Street Tower, 8th Floor, San Francisco, CA, 94105, US

(73) Assignee: Nektar Therapeutics, San Carlos, CA (US)

(21) Appl. No.: 11/296,108

(22) Filed: Dec. 6, 2005

Related U.S. Application Data
(60) Provisional application No. 60/633,988, filed on Dec. 7, 2004.

(51) Int. Cl. A61K 31/4184

(52) U.S. Cl. 514/381

(57) ABSTRACT

One or more embodiments of the invention provide various novel formulations, and tablet dosage forms, comprising losartan that are non-crystalline, stable, and/or otherwise improvements over known losartan formulations. One or more embodiments of the invention further provide methods for preparing the formulation, methods for preparing the tablet dosage form, and to methods of administering the tablet dosage and/or formulation comprising losartan. The losartan-containing formulations may be administered to a user to treat hypertension, and related conditions.

### RELATED APPLICATION

This application relates to U.S. Provisional Application No. 60/633,988, filed Dec. 7, 2004, from which priority is claimed under 35 USC §119(e), and which is incorporated herein by reference in its entirety.

### BACKGROUND OF THE INVENTION

One of more embodiments of the present invention relate to a formulation comprising losartan, to methods for preparing the formulation, to a tablet dosage form of the losartan, to methods for preparing the tablet dosage form, and to methods of administering the tablet dosage and/or formulation comprising losartan.

Losartan, 2-butyl-4-chloro-1-[(2′-tetrazol-5-yl)-biphenyl-4-yl]methyl]-5-(hydroxymethyl)imidazole, is a well known pharmaceutical agent. U.S. Pat. Nos. 5,138,069 and 5,153,197 both to Carini et al., both of which are incorporated herein by reference in their entireties, describe the angiotensin II blocking ability of certain substituted imidazoles, such as losartan, and the effectiveness of the compounds in treating hypertension and/or congestive heart failure. Losartan has also been determined to be effective in treating renal failure, as described in U.S. Pat. No. 5,210,079 to Carini et al., which is incorporated herein by reference in its entirety.

Losartan is commercially available in the United States from Merck & Co., Inc. in Whitehouse Station, N.J., under the tradename COZAAR®. According to the Merck product description, COZAAR® is an angiotensin II receptor (type AT₁) antagonist comprising losartan in the form of its potassium salt, chemically described as 2-butyl-4-chloro-1-[p-(o-1H-tetrazol-5-ylphenyl)benzyl]imidazole-5-methanol monopotassium salt, with the empirical formulation C₂₂H₂₂ClKN₆O and with the structural formula:

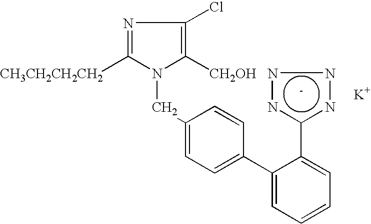

COZAAR® is available as orally administrable tablets in the following dosage amounts: 25 mg, 50 mg, and 100 mg of losartan potassium. The COZAAR® tablets also contain the following inactive ingredients: microcrystalline cellulose, lactose hydrous, pregelatinized starch, magnesium stearate, hydroxypropyl cellulose, hypromellose, titanium dioxide, D&C yellow No. 10 aluminum lake and FD&C blue No. 2 aluminum lake.

Losartan potassium may be administered alone or in combination with other active agents. For example, losartan potassium is also available from Merck & Co., Inc. under the tradename HYZAAR®, which is a tablet formulation combining losartan potassium with a diuretic. As described in the Merck product description, HYZAAR® combines losartan potassium, as described above, with hydrochlorothiazide, which is chemically described as 6-chloro-3,4-dihydro-2H-1,2,4-benzothiadiazine-7-sulfonamide 1,1-dioxide. The empirical formula for hydrochlorothiazide is C₇H₈ClN₃O₄S₂ and the structural formula is

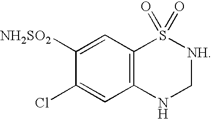

HYZAAR® is available in the following dosages: 50 mg of losartan potassium with 12.5 mg of hydrochlorothiazide and 100 mg of losartan potassium with 25 mg of hydrochlorothiazide. The inactive ingredients in the HYZAAR® Tablet are identified as microcrystalline cellulose, lactose hydrous, pregelatinized starch, magnesium stearate, hydroxypropyl cellulose, hypromellose, titanium dioxide and D&C yellow No. 10 aluminum lake.

Description of Related Art

In both the COZAAR® and HYZAAR® tablets, the losartan potassium is in a crystalline form. Crystalline polymorphic Forms I and II are described in U.S. Pat. No. 5,608,075 which is incorporated herein by reference in its entirety. U.S. Pat. No. 5,608,075 describes various methods for making the crystalline polymorphic forms of losartan potassium, the mechanism for losartan potassium's action, and various dosage forms comprising the crystalline polymorphic forms of losartan potassium.

U.S. Pat. No. 5,608,075 (and corresponding PCT Application Publication WO95/17396) to Campbell et al describes distinct crystalline structures, or forms, of losartan potassium. U.S. Pat. No. 5,140,037 to Chiu et al. discloses conventional crystalline forms of imidazole angiotensen-II receptor antagonists for the treatment of impaired cognitive performance.

The above-described crystalline forms of losartan have disadvantages. The crystalline forms of losartan are physically stable in that they do not easily convert to another form during storage or processing, however, the crystalline forms generally have poorer dissolution rates than those of non-crystalline forms. While the free acid form of losartan is not very soluble, the potassium salt form has acceptable solubility. The non-crystalline forms, however, often have increased bioavailability when administered to a user because of their ability to dissolve faster in the GI tract, as recognized in the art. This increased bioavailability can allow for the active agent to be taken up faster for systemic delivery. Also, the increased bioactivity can allow for a reduction in the amount of the active agent that needs to be administered to the user. The formulation of non-crystalline losartan potassium has been attempted with only limited success. For example, U.S. Patent Application Publication 2004-0006237 to Dolitzky (and corresponding PCT Application Publication WO 03/048135), both of which are incorporated herein by reference in their entireties, describe an amorphous form of losartan potassium produced by dissolving losartan potassium in a solvent and removing the solvent from the solution in a manner that forms pure amorphous losartan potassium. However, when pure amorphous losartan potassium is formulated as described in WO 03/048135 the formulation has limited physical stability. Under normal storage conditions, the pure amorphous losartan potassium tends to alter its form and often converts to one or more of its crystalline forms. Because the degree of crystalline conversion at a particular time during the storage is often unknown, it is difficult to assure that dosages are administered in a consistent solid form. As a result, the losartan must either be administered immediately after formulation or a sufficient amount of storage time must pass so that full conversion to a crystalline form takes place, in which case the advantages of having the losartan in amorphous form are lost. Moreover, the publications do not teach, suggest or disclose a preparation of amorphous losartan with an excipient, nor such a preparation having stability properties comparable to commercially-available crystalline losartan. The Dolitzky publications also do not teach a method of preparing wherein a particulate product results (other than through the use of a separate milling step).

PCT Application WO 2004/064834 to Kumar et al., describes spray-drying losartan plus a polymer to form what is said to be an amorphous particle. Kumar et al., does not teach a tablet dosage form of the powder. The particles disclosed in this reference are limited to those formed by spray-drying, and which have the specified vinylpyrrolidone polymer. Moreover, Kumar et al. is silent as to a physical properties of the particles, such as size, density, size distribution as well as the formulations' dissolution stability and chemical stability.

Therefore, it is desirable to be able to produce an improved non-crystalline form of losartan. It is further desirable to be able to produce a non-crystalline form of losartan that maintains its non-crystalline state for an increased amount of time when compared to pure amorphous losartan. Non-crystalline forms may have other advantages, such as handling and/or tableting advantages.

### SUMMARY OF THE INVENTION

One or more embodiments of the present invention satisfies these needs. The invention provides various novel formulations comprising losartan that are non-crystalline, more stable, and/or otherwise improvements over known losartan formulations.

In one aspect of the invention, a solid, non-crystalline formulation comprises losartan wherein the formulation is physically stable.

In another aspect of the invention, a solid, non-crystalline formulation comprises losartan wherein the formulation maintains its non-crystalline form when stored at 25° C. and 60% relative humidity for a period of at least 1 week, more preferably at least 1 month, more preferably at least one year.

In another aspect of the invention, a solid, non-crystalline formulation comprises losartan wherein the formulation maintains its non-crystalline form when stored at 40° C. and 75% relative humidity for a period of at least 1 week, more preferably at least 1 month, more preferably at least three months.

In one aspect of the invention, a solid non-crystalline formulation comprises losartan potassium wherein the formulation exhibits at least one of the characteristics of acceptable, or parity dissolution, solubility, stability, shelf life or bioavailability, when compared to a commercially-available formulation.

In one aspect of the invention, a solid, non-crystalline formulation comprises losartan and an excipient, wherein the formulation exhibits at least one of the characteristics of enhanced dissolution, solubility, stability, shelf life, bioavailability, or tabletting ease or manufacturing cost-effectiveness.

In another aspect of the invention, a solid, non-crystalline formulation comprises particles, wherein the particles comprise losartan and an excipient.

In another aspect of the invention, a solid, non-crystalline formulation comprises particles, wherein the particles comprise losartan and an excipient, and wherein the excipient comprises a co-polymer of vinyl pyrrolidone and vinyl acetate.

In another aspect of the invention, a solid, non-crystalline formulation comprises particles, wherein the particles comprise losartan and an excipient, and wherein the excipient comprises a co-polymer of vinyl pyrrolidone and vinyl acetate, wherein a ratio of vinyl pyrrolidone:vinyl acetate is between about 8:2 to 2:8.

In another aspect of the invention, a solid, non-crystalline formulation comprises particles, wherein the particles comprise losartan and an excipient, and wherein the excipient comprises a co-polymer of vinyl pyrrolidone and vinyl acetate, wherein a ratio of vinyl pyrrolidone:vinyl acetate is about 6:4.

In another aspect of the invention, a solid, non-crystalline formulation comprises particles, wherein the particles comprise losartan and a stabilizing excipient, wherein the formulation is more physically stable than a formulation without the stabilizing excipient.

In another aspect of the invention, a solid, non-crystalline formulation comprises particles, wherein the particles comprise losartan and a stabilizing excipient, wherein the formulation when stored at 40° C. and 75% relative humidity converts to a crystalline form more slowly than a formulation without the stabilizing excipient.

In another aspect of the invention, a solid, non-crystalline formulation comprises particles, wherein the particles comprise losartan and a stabilizing excipient, wherein the formulation has a higher glass transition temperature than a formulation without the stabilizing excipient.

In another aspect of the invention, a solid, non-crystalline formulation comprises particles, wherein the particles comprise losartan and a stabilizing excipient, wherein the formulation has a glass transition temperature of above about 40° C.

In another aspect of the invention, a solid formulation comprises a tablet dosage form, wherein the tablet comprises non-crystalline losartan and a stabilizing excipient.

In another aspect of the invention, a solid formulation comprises a tablet dosage form, wherein the tablet comprises non-crystalline losartan and a stabilizing excipient and wherein the tablet contains no binder.

In another aspect of the invention, a solid formulation comprises a tablet dosage form, wherein the tablet comprises non-crystalline losartan and a stabilizing excipient and wherein the tablet contains no disintegrant.

In another aspect of the invention, a solid formulation comprises a tablet dosage form, wherein the tablet comprises non-crystalline losartan and a stabilizing excipient and wherein the tablet contains no binder or disintegrant, and which provides bioavailability at least parity with that of a commercially-available product.

In another aspect of the invention, a method of treating hypertension comprises administering to a user a non-crystalline formulation comprising losartan.

In another aspect of the invention, a method of treating hypertension comprises administering to a user a non-crystalline formulation comprising losartan following storage of the non-crystalline formulation.

In another aspect of the invention, a method of treating hypertension comprises administering to a user a particulate formulation wherein the particles comprise non-crystallin losartan and an excipient.

In another aspect of the invention, a method of treating hypertension comprises administering to a user a non-crystalline, particulate formulation wherein the particles comprise losartan and a stabilizing excipient.

In another aspect of the invention, a method of making a formulation comprising losartan comprises providing a liquid containing losartan and spray drying the liquid under conditions appropriate to produce particles comprising non-crystalline losartan which exhibits acceptable solubility and/or bioavailability.

In another aspect of the invention, a method of making a formulation comprising losartan comprises providing a liquid comprising losartan and contacting liquid with a supercritical or near critical fluid to remove the solvent form the liquid to produce particles comprising non-crystalline losartan.

In another aspect of the invention, a method of making a formulation comprising losartan comprises providing an aqueous liquid containing losartan and an excipient and removing the aqueous liquid to produce particles comprising losartan and the excipient.

In another aspect of the invention, a method of making a formulation comprising losartan comprises providing an aqueous liquid containing losartan and an excipient and removing the aqueous liquid to produce particles comprising non-crystalline losartan and the excipient wherein the particles exhibit at least one of the characteristics of parity or enhanced dissolution, solubility, stability, shelf life, or bioavailability when compared to a commercially-available product, or tabletting ease or manufacturing cost-effectiveness.

In another aspect of the invention, a method of making a formulation comprising losartan comprises providing an organic solvent containing losartan and removing the organic solvent to produce particles comprising losartan.

In another aspect of the invention, a method of making a formulation comprising losartan comprises providing an organic solvent containing losartan and an excipient and removing the organic solvent to produce particles comprising losartan and the excipient.

In another aspect of the invention, a method of making a formulation comprising losartan comprises spray drying a liquid containing losartan and an excipient to produce particles comprising non-crystalline losartan and the excipient.

In another aspect of the invention a method of making a formulation comprising losartan comprises providing a liquid containing losartan free acid and adding a salt comprising an alkali earth metal or an alkaline earth metal and a counter ion. The liquid is then removed to form a non-crystalline losartan salt.

In another aspect of the invention a method of making a formulation comprising losartan comprises providing water and adding to the water losartan free acid and a substantially equal mole of potassium hydroxide. The water is then removed to form non-crystalline losartan potassium.

In another aspect of the invention a method of making an immediate-release tablet comprising non-crystalline losartan comprises forming an intimate mixture of losartan and excipient, and compacting into a tablet.

In another aspect of the invention, any two or more of the above aspects are combined.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of a particular example or drawing, and the invention includes any combination of these features, where:

FIG. 1A is a graph showing an X-ray powder diffraction (XRPD) profile for a prior art form of losartan in its crystalline polymorphic Form 1;

FIG. 1B is a graph showing an X-ray powder diffraction (XRPD) profile for prior art form of losartan in its crystalline polymorphic Form 2;

FIG. 1C is a graph showing an X-ray powder diffraction (XRPD) profile for a prior art form of commercially-available losartan;

FIG. 1D is polarized light photomicrograph of a prior art form of commercially-available losartan, the photomicrograph taken soon after formulation;

FIG. 2 is a schematic block diagram of one embodiment of a spray-drying process according to the present invention;

FIG. 3 is a schematic diagram of an embodiment of an apparatus for carrying out a spray-drying process according to the present invention;

FIG. 4 is a schematic diagram of one embodiment of an apparatus for carrying out a particle precipitation process according to the present invention;

FIG. 5A is a graph showing an X-ray powder diffraction (XRPD) profile for particles comprising non-crystalline losartan potassium and a stabilizing excipient produced by removing an aqueous solvent from a solution containing the losartan potassium and the stabilizing excipient, in accordance with one or more aspects of the present invention;

FIG. 5B is a graph showing an X-ray powder diffraction (XRPD) profile for the formulation analyzed in FIG. 5A after the formulation was exposed to 75% relative humidity at 40° for 1 week;

FIG. 6A shows a polarized light photomicrograph of the formulation analyzed in FIG. 5A;

FIG. 6B shows a polarized light photomicrograph of the formulation analyzed in FIG. 6A after the formulation was exposed to 75% relative humidity at 40° for 1 month;

FIG. 7 is a graph of a moisture sorption isotherm showing the glass transition temperature, T_{g}, of the formulation analyzed in FIG. 5A as a function of relative humidity;

FIG. 8A is a graph showing an X-ray powder diffraction (XRPD) profile for particles comprising non-crystalline losartan potassium and a stabilizing excipient produced by making a solution comprising losartan potassium and stabilizing excipient in an organic solvent, and removing the organic solvent by contacting the solution with a supercritical or near critical antisolvent, in accordance with one or more aspects of the present invention;

FIG. 8B is a graph showing an X-ray powder diffraction (XRPD) profile for the formulation analyzed in FIG. 8A after the formulation was exposed to 75% relative humidity at 40° for 1 month;

FIG. 9 is a DSC thermogram of the specific heat as a function of temperature for pure non-crystalline losartan particles (absent excipient) made by a supercritaical solvent removal process technique of the present invention;

FIG. 10 is a DSC thermogram of the specific heat as a function of temperature for the formulation analyzed in FIGS. 8;

FIG. 11 is a graph showing an X-ray powder diffraction (XRPD) profile for another version of particles comprising non-crystalline losartan potassium and a stabilizing excipient produced by making a solution comprising losartan potassium and stabilizing excipient in an organic solvent, and removing the organic solvent by contacting the solution with a supercritical or near critical antisolvent, in accordance with one or more aspects of the present invention, the graph showing the profile after the formulation was exposed to 75% relative humidity at 40° for 1 month;

FIG. 12 is a DSC thermogram of the specific heat as a function of temperature for the formulation analyzed in FIG. 11;

FIG. 13 is a graph showing an X-ray powder diffraction (XRPD) profile for another version of particles comprising non-crystalline losartan potassium and a stabilizing excipient produced by making a solution comprising losartan potassium and stabilizing excipient in an organic solvent, and removing the organic solvent by contacting the solution with a supercritical or near critical antisolvent, in accordance with one or more aspects of the present invention, the graph showing the profile after the formulation was exposed to 75% relative humidity at 40° for 1 month;

FIG. 14 is a DSC thermogram of the specific heat as a function of temperature for the formulation analyzed in FIG. 13.

FIG. 15 is a DSC thermogram of the specific heat as a function temperature for another version of particles comprising non-crystalline losartan potassium and a stabilizing PVPVA excipient produced by spray-drying in accordance with one or more embodiments herein, and made by starting with the free acid form of losartan;

FIG. 16 is is an X-ray powder diffraction (XRPD) profile for the particles comprising non-crystalline losartan potassium and a stabilizing PVPVA excipient, produced by spray-drying, analyzed in FIG. 15;

FIG. 17 is a graph of particle size distribution for an example of bulk powder particles comprising non-crystalline losartan potassium and a stabilizing PVPVA excipient produced by making a solution comprising losartan potassium and PVPVA excipient in water, and removing the water by spray drying in accordance with one or more aspects of the present invention, the bulk powder formulation made in accordance with a process of the present invention;

FIG. 18 is a SEM photomicrographic image of the powder formulation made accordance with a process of the present invention, the powder representing that for which the particle size distribution is shown in FIG. 17;

FIG. 19 is a thermal gravimetric plot of the powder of FIGS. 17 and 18, showing a water loss of about 5%;

FIG. 20 is a dissolution profile of a tablet dosage formulation comprising a powder manufactured using one or more processes of the present invention, the tablet dosage form made by dry granulation (using a roller compactor) and granules compacted into tablets, the curves in FIG. 20 representing initial dissolution (square label); dissolution after 1 month at 25° C./60% RH (triangle label); dissolution after 2 months at 25° C./60% RH (diamond label); and dissolution after 3 months at 25° C./60% RH (asterisk label);

FIG. 21A is a drug concentration in human plasma time plot showing a losartan tablet formulation made in accordance with one or more methods of the present invention, and a metabolite comprising losartan 5-carboxylic acid, compared with a commercially-availabe prior art formulation (as COZAAR®). Drug concentration (in ng/mL) is plotted against post dose time; and

FIG. 21B is drug concentration in human plasma time plot showing a losartan tablet formulation made in accordance with one or more methods of the present invention, and a metabolite comprising losartan 5-carboxylic acid, compared with a commercially-availabe prior art formulation (as COZAAR®). The log of drug concentration (in ng/mL) is plotted against post dose time.

### DETAILED DESCRIPTION OF SPECIFIC EMBODIMENTS

One or more embodiments of the present invention relates to a formulation comprising losartan, to a method of making a formulation comprising losartan, and to a method of administering a formulation comprising losartan. One or more embodiments of the present invention further relates to a pharmaceutical composition comprising losartan, to a method of making a pharmaceutical composition comprising losartan, and to a method of administering a pharmaceutical composition comprising losartan. Although the invention is illustrated in the context of a particulate formulation, the present invention can be used in other forms and for purposes other than for those specifically disclosed, and the invention should not be limited to the examples provided herein.

Definitions

Before describing the present invention in detail, it is to be understood that the invention is not limited to the particularly exemplified apparatus, systems, methods, or processes disclosed herein, which may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include the plural unless the content clearly dictates otherwise.

Reference herein to “one embodiment”, “one version” or “one aspect” shall include one or more such embodiments, versions or aspects, unless otherwise clear from the context.

Unless defined otherwise, all technical and scientific terms used herein have the same meaning as commonly understood by one of ordinary skill in the art to which the invention pertains. A number of methods and materials similar or equivalent to those described herein can be used in the practice of the present invention.

Amount of ingredients, materials or substances are listed as the ranges or levels of ingredients in the descriptions, which follow hereto.

“Therapeutically-effective amount” means that amount of active present in the composition that is needed to provide the desired level of drug in the subject to be treated to yield the expected physiological response.

“Drug” means any compound or composition which induces a desired pharmacologic and/or physiologic effect, when administered appropriately to the target organism (human or animal). Losartan is one example of a drug.

The term “vehicle” means a fluid which dissolves a solid or solids, to form a solution, or which forms a suspension of a solid or solids which do not dissolve or have a low solubility in the fluid. The vehicle can be composed of one or more fluids.

As used herein, a ‘co-formulation’ refers to two or more substances formulated at substantially the same time and/or formulated so that a particle comprising a co-formulation contains the two or more substances. For example, a co-formulation may comprise a solid dispersion of a first substance and a second substance, such as an intimate mixture of an active substance and an excipient. In one version, the intimate mixture may comprise an active agent, especially a pharmaceutically-active agent, such as losartan, dispersed in a “matrix” of a carrier material, especially an excipient, such as an oligomeric and/or polymeric excipient. The co-formulations of one or more embodiments of the present invention with an excipient may advantageously modify the solubility and/or dissolution characteristics of the active substance. Unless otherwise clear from the context, a formulation includes a co-formulation.

By “losartan” it is meant the compound 2-butyl-4-chloro-1-[(2′-tetrazol-5-yl)-biphenyl-4-yl]methyl]-5-(hydroxymethyl) imidazole and comprises all compounds having any of the following chemical formulas: 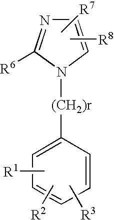
wherein
R¹ 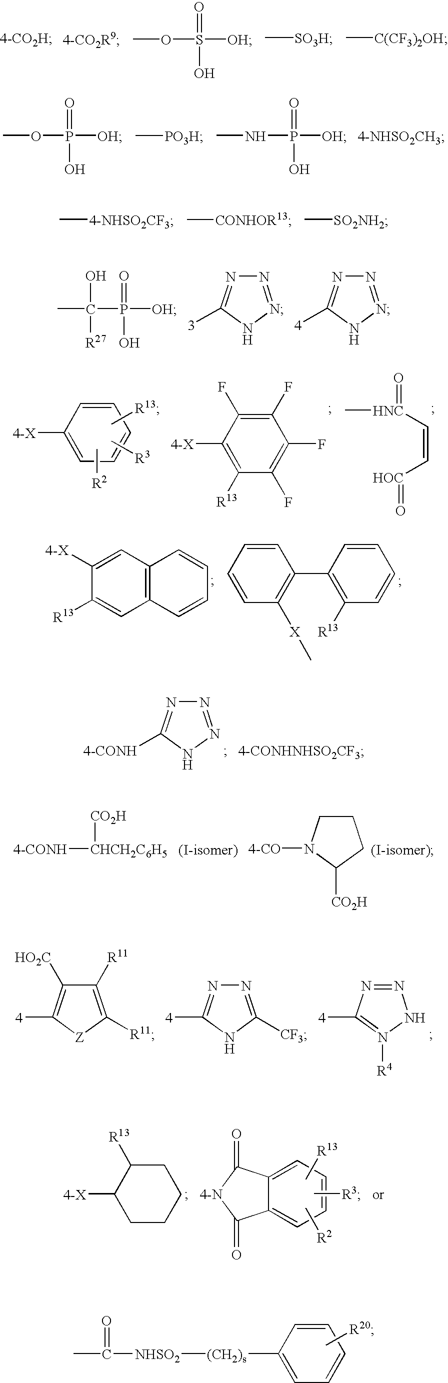
R² is H; Cl; Be; I; F; NO₂; CN; alkyl of 1 to 4 carbon atoms; acyloxy of 1 to 4 carbon atoms; alkoxy of 1 to 4 carbon atoms; CO₂H; CO₂R⁹; NHSO₂CH₃; NHSO₂CF₃; 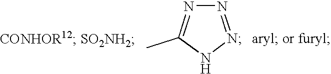
R³ is H; Cl, Br, I or F; alkyl of 1 to 4 carbon atoms or alkoxy of 1 to 4 carbon atoms;
R⁴ is CN, NO₂ or CO₂R¹¹;
R⁵ is H, alkyl of 1 to 6 carbon atoms, cycloalkyl of 3 to 6 carbon atoms alkenyl or alkynyl of 2 to 4 carbon atoms;
R⁶ is alkyl of 2 to 10 carbon atoms, alkenyl or alkynyl of 3 to 10 carbon atoms of the same groups substituted with F or CO₂R¹⁴; cycloalkyl of 3 to 8 carbon atoms, cycloalkylalkyl of 4 to 10 carbon atoms; cycloalkylalkenyl or cycloalkylalkynyl of 5 to 10 carbon atoms (CH₂)₃Z(CH₂) [symbol] R³ optionally substituted with F or CO₂R¹⁴; benzyl substituted on the phenyl ring with 1 or 2 halogens, alkoxy of 1 to 4 carbon atoms, alkyl of 1 to 4 carbon atoms or nitro; R⁷ is H, F, Cl, Br, I, NO₂, C [symbol] F_{2v+L}, where v=1-6, C₆F₅; CN; 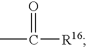
   straight or branched alkyl of 1 to 6 carbon atoms; phenyl or phenylalkyl, where alkyl is 1 to 3 carbon atoms; or substituted phenyl or substituted phenylalkyl, where alkyl is 1 to 3 carbon atoms, substituted with one or two substituents selected from alkyl of 1 to 4 carbon atoms, F, Cl, Br, OH, OCH₃, 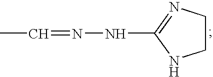
R⁹ is 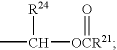
R¹⁰ is alkyl of 1 to 6 carbon atoms or perfluoroalkyl of 1 to 6 carbon atoms, 1-adamantyl, 1-naphthyl, 1-(1-naphthyl)ethyl, or (CH₂) [symbol] C₆H₅;
R¹¹ is H, alkyl of 1 to 6 carbon atoms, cycloalkyl of 3 to 6 carbon atoms, phenyl or benzyl;
R¹² is H, methyl or benzyl;
R¹³ is —CO₂H; —CO₂H; —CO₃R⁹; —CH₃CO₂H, —CH₂CO₂R⁹; 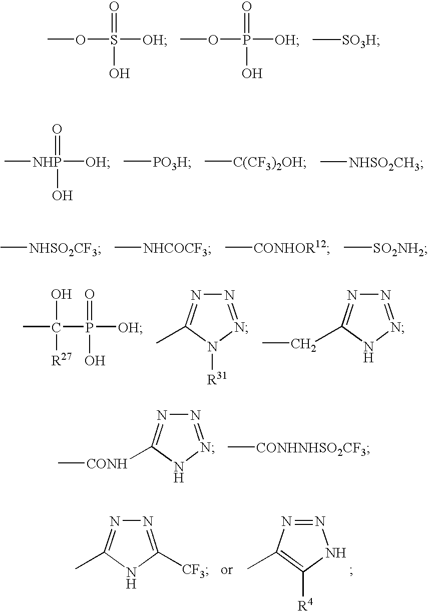
R¹⁴ is H, alkyl or perfluoroalkyl of 1 to 8 carbon atoms, cycloalkyl of 3 to 6 carbon atoms, phenyl or benzyl;
R¹⁵ is H, alkyl of 1 to 6 carbon atoms, cycloalkyl of 3 to 6 carbon atoms, (CH₂) [symbol] C₆H₅, OR¹⁷, or NR¹⁸R¹⁹;
R¹⁷ is H, alkyl of 1 to 6 carbon atoms, cycloalkyl of 3 to 6 carbon atoms, phenyl or benzyl;
R¹⁸ and R¹⁹ independently are H, alkyl of 1 to 4 carbon atoms, phenyl, benzyl, α-methylbenzyl, or taken together with the nitrogen form a ring of the formula 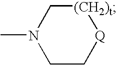
Q is NR²⁰, O or CH₃;
R²⁰ is H, alkyl of 1-4 carbon atoms, or phenyl;
R²¹ is alkyl of 1 to 6 carbon atoms, —NR²²R²³, 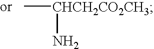
R²² and R²³ independently are H, alkyl of 1 to 6 carbon atoms, benzyl, or are taken together as (CH₂) [symbol] where [symbol] is 3-6;
R²⁴ is H, CH₃ or —C₆H₅;
R²⁵ is NR²⁷R²⁸, OR²⁸, NHCONH₂, NHCSNH₂, 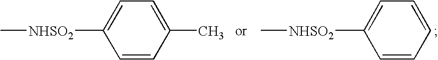
R²⁶ is hydrogen, alkyl with from 1 to 6 carbon atoms, benzyl, or alkyl;
R²⁷ and R²⁸ are independently hydrogen, alkyl with from 1 to 5 carbon atoms, or phenyl;
R²⁹ and R³⁰ are independently alkyl of 1-4 carbon atoms or taken together are —(CH₃) [symbol] —;
R³¹ is H, alkyl of 1 to 4 carbon atoms, —CH₂CH═CH₂ or —CH₂C₆H₄R³²;
R³² H, NO₂, NH₂, OH or OCH₃;
X is a carbon-carbon single bond, —CO—, —CH₃—, —O—, —S—, 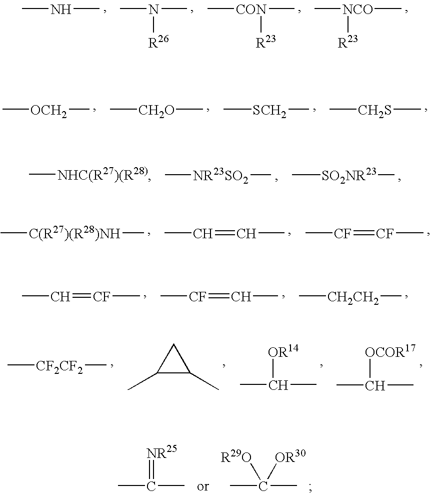
Y is O or S;
Z is O, NR¹¹, or S;
m is 1 to 5;
n is 1 to 10;
p is 0 to 3;
q is 2 to 3;
r is 0 to 2;
s is 0 to 5;
t is 0 to 1;
   and pharmaceutically acceptable salts of these compounds;
provided that:
(1) the R¹ group is not in the ortho position;
(2) when R¹ is 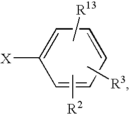
X is a single bond, and R¹³ is CO₂H, or 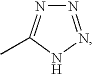
   then R¹³ must be in the ortho or meta position; or when R¹ and X are as above and R¹³ is NHSO₂CF₃ or NHSO₂CH₃, R¹³ must be ortho;
(3) when R¹ is 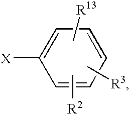
   and X is other than a single bond, then R¹³ must be ortho except when X═NR²³CO and R¹³ is NHSO₂CF₃ or NHSO₂CH₃, then R¹³ must be ortho or meta;
(4) when R¹ is 4-CO₂H or a salt thereof, R⁶ cannot be S-alkyl;
(5) when R¹ is 4-CO₂H or a salt thereof, the substituent on the 4-position of the imidazole cannot be CH₂OH, CH₂COCH₃, or CH₂CO₂H;
(6) when R¹ is 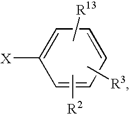
X is —OCH₂—, and R¹³ is 2-CO₂H, and R⁹ is H then R⁶ is not C₂H₃S;
(7) when R¹ is 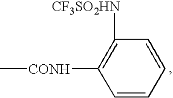
   and R⁶ is n-hexyl then R⁷ and R⁸ are not both hydrogen;
(8) when R¹ is 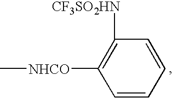
R⁶ is not methoxybenzyl;
(9) the R⁶ group is not 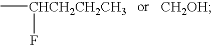
(10) when r=0m R¹ is 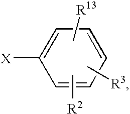
X is 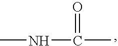
R¹³ is 2-NHSO₂CH₃, and R⁶ is n-propyl, then R⁷ and R⁸ are not —CO₂CH₃;
(11) when r=0, R¹ is 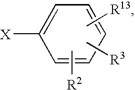
X is 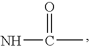
R¹³ is 2-COOH, and R⁶ is n-propyl, then R³ and R² are not —CO₂CH₃;
(12) when r−1, R¹═ 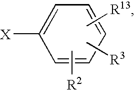
X is a single bond, R⁷ is Cl, and R⁸ is —CHO, then R¹³ is not 3-(tetrasol-5-yl);
(13) when r=1, R³═ 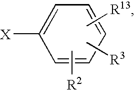
X is a single bond, R⁷ is Cl, and R⁸ is —CHO, then R¹³ is not 4-(tetrasol-5-yl);
(14) when r=0, then R¹ is not 4-NHSO₂CH₃ or 4-NHSO₂CF₃.

and which have angiotensin II-antagonizing properties and/or are useful as antihypertensive agents. The losartan compound may be in its free acid form or in the form of any pharmaceutically acceptable salt, ester, or prodrug of losartan. The term “pharmaceutically acceptable salts” includes, but is not limited to, alkali metal or alkaline earth metal salts such as sodium, potassium, calcium, lithium, magnesium, zinc or the like.

By “losartan potassium” it is meant the monopotassium salt of losartan, as shown by the structural formula:

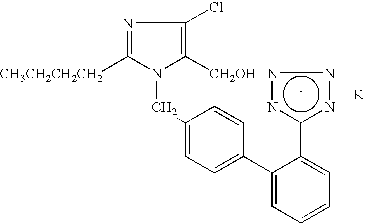

By “crystalline” it is meant any solid which gives a wide angle x-ray powder diffraction pattern showing one or more characteristic peaks that result from the solid's three dimensional structure, including pure compounds and mixtures which show such peaks. The x-ray powder diffraction may be performed by any suitable instrument, such as a D5000 XRD (Siemens, Germany) between 2 and 40° 2θ, at a scan rate of 0.02 degrees per second.

By “non-crystalline” it is meant any solid which does not give rise to one or more characteristic peaks in wide angle x-ray powder diffraction indicative of crystallinity as defined above. This includes amorphous materials, which are disordered at the molecular level, and liquid crystals, such as frozen thermotropic liquid crystals, which can be distinguished from amorphous materials because they exhibit birefringence under polarized light, and microcrystalline forms which do not give rise to one or more characteristic peaks in wide angle x-ray diffraction. “Non-crystalline” also includes pure amorphous materials and amorphous mixtures of materials. In the case of a mixture, this includes molecular solid dispersions, which are comparable to liquid solutions in that there is a single phase which is disordered at the molecular level, non-molecular solid dispersions, which have one or more distinct amorphous phases, and to other homogeneous or non-homogeneous mixtures, provided there is no crystallinity as defined above.

One or more embodiments of the present invention provide an improved formulation comprising losartan. Among other improvements, the losartan-containing formulation described herein offers improvements over prior art formulations containing crystalline losartan in that the present formulation provides losartan in a form where it has a dissolution rate which provides a desired, especially a commercially-desired, bioavailability. Additionally or alternatively, the present formulation is advantageous over known pure amorphous forms of losartan in that the present formulation has improved processability and/or improved physical stability and/or improved chemical stability, allowing the present formulation to be stored over longer periods of time and/or allowing the formulation more time for being processed into a solid dosage form, such as a tablet.

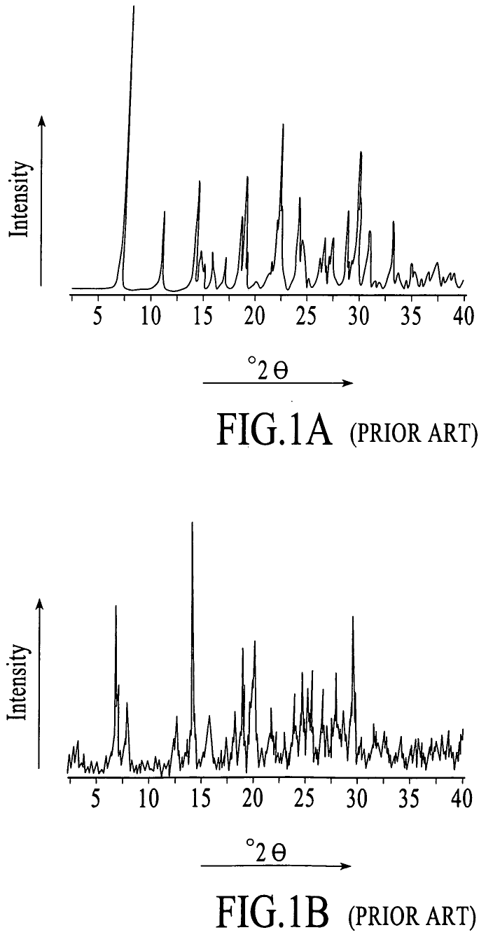

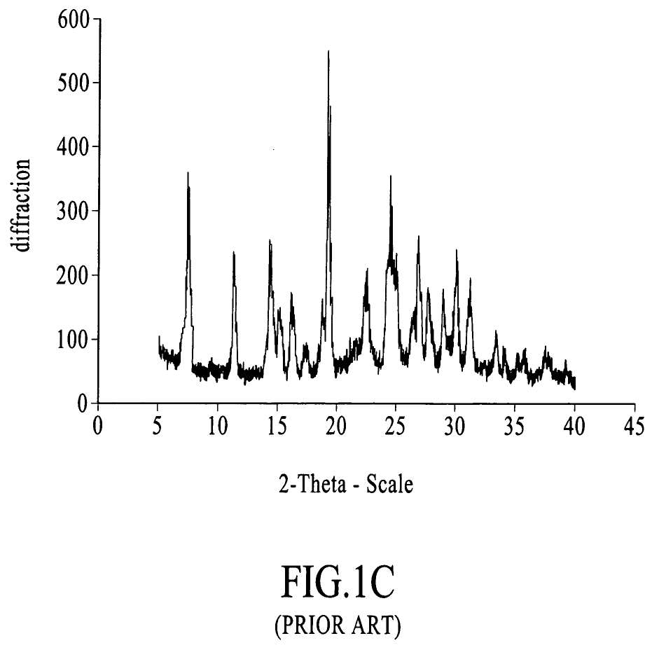

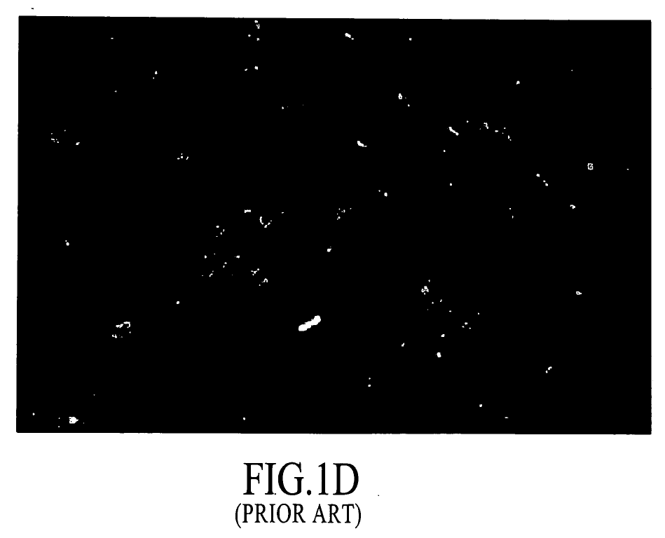

Solid losartan is conventionally present in one or more of its stable crystalline polymorphic forms. For example, as disclosed in U.S. Pat. No. 5,608,075, losartan may be processed to be in either crystalline polymorphic Form 1 or crystalline polymorphic Form 2. Each of these crystalline polymorphic forms may be characterized by analyzing the X-ray powder diffraction pattern of the solid material. FIG. 1A shows the X-ray powder diffraction pattern disclosed in U.S. Pat. No. 5,608,075 for crystalline polymorphic Form 1 of losartan. Form 1 may be characterized by having the following powder diffraction angles: 7.24, 11.02, 14.16, 15.07, 18.46, 18.87, 26.53, 27.30, and 29.15. FIG. 1B shows the X-ray powder diffraction pattern disclosed in U.S. Pat. No. 5,608,075 for crystalline polymorphic Form 2 of losartan. Form 2 may be characterized by having the following powder diffraction angles: 2.95, 6.95, 7.91, 12.61, 14.28, 18.98, 20.01, 21.63, and 29.15. Commercially available losartan, supplied by Sai Life Sciences Limited in Hyderabad, India, has been tested and analyzed and has the X-ray powder diffraction pattern shown in FIG. 1C. From observing the pattern shown in Figure IC, it can be seen that the commercially available losartan is at least partially in crystalline form. The crystalline form of the commercially available losartan is further verified by the polarized light micrograph of the commercially available losartan shown in FIG. 1D.

As discussed above, the crystalline form of losartan has proven to be stable and effective. However, when in the non-crystalline form, the losartan has a dissolution rate and/or profile that is higher than when the losartan is in a crystalline form. Accordingly, in one or more versions of the present invention, a formulation comprising losartan is provided in non-crystalline form. By providing non-crystalline losartan, the efficacy of the losartan is maintained while the desired dissolution rate is attained, thereby providing an improved form of the pharmaceutical agent. In one or more embodiments, the desired dissolution rate and/or profile is substantially equal to, or parity with a commercially-available product, such as COZAAR® 100 mg tablets. In other embodiments, the desired dissolution rate and/or profile is better than a commercially-available product, such as COZAAR® 100 mg tablets.

In one or more versions, the non-crystalline formulation is produced by spray drying. During the spray drying process the losartan is dissolved or suspended within a liquid. This mixture is then passed through a nozzle, or other atomizer, which introduces droplets of the mixture into a chamber. As the droplets dry, the liquid is removed thereby producing solid particles comprising non-crystalline losartan. The particles are then collected, such as by filtration or cyclone separation, to provide a particulate composition that may be administered to a user or further processed into a dosage form.

By “spray drying” it is meant the process of producing a particulate solid from a solution, slurry, emulsion, or suspension, or the like, of the losartan in a liquid, such as an aqueous or organic liquid, by atomizing the liquid to form droplets and drying the droplets to form a particulate solid. Generally, the particles have a moisture content of less than about 10% by weight water, preferably less than about 5% by weight water and sometimes less than about 3% by weight water, and may be from about 3% to about 5%. The drying conditions are suitably chosen to provide the desired moisture levels. The particle size (mass mean diameter) may be tailored to be a particular size as dictated by the end usage. For tableting, the size may be about 10 to about 500 μm, and in one or more versions is in the range of about 10 to about 200 μm, or about 20 to about 100 μm, or about 20 to about 50 μm. Smaller particle sizes, for example about 10 μm or less, or larger particle sizes, for example about 500 or greater, may have applications in additional or alternative dosage forms.

During the spray drying process, atomization of the liquid may be performed using a conventional atomizer such as a centrifugal, sonic, pressure and/or rotary atomizer. In one or more versions, a rotary atomizer is used in which the liquid flows over the wheel surface as a thin film, and is sheared away into discrete droplets. Other suitable atomizers include two-fluid atomizers, wherein liquid and atomization gas stream are delivered concurrently. Typically, the atomization gas is pressurized to high pressure for delivery through an atomization nozzle. Often the gas is air although other gases such as nitrogen may also be used. An example of a suitable spray drying method is a method as described in The Spray Drying Handbook, by Keith Masters, Longman Publishing, 5th Ed., September 1991, the contents of which is incorporated herein by reference in its entirety. Other spray-drying references include U.S. Pat. No. 6,592,904 and/or WO 03/037303, the contents of which are incorporated herein by reference in their entireties.

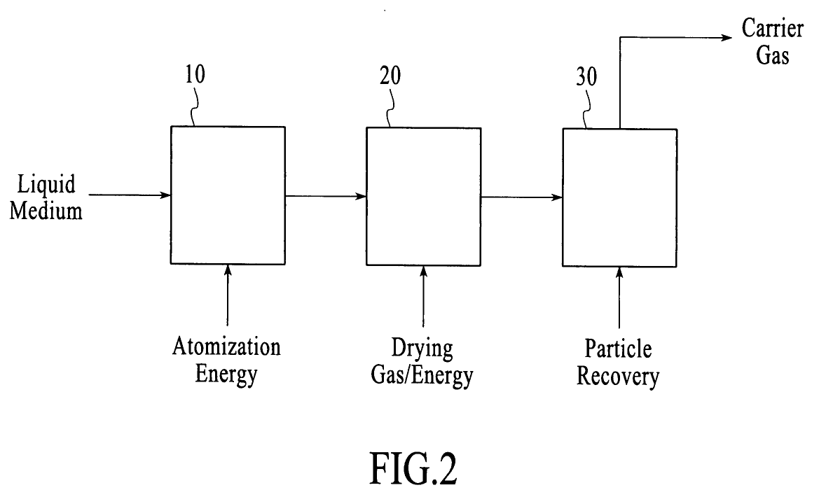

In one or more embodiments of the present invention, and referring to FIG. 2, a spray-drying process comprises an atomization operation 10 that produces droplets of a liquid medium, which are subsequently dried in a drying operation 20. The drying operation 20 may be a single drying chamber or a multi-stage operation. Drying of the liquid droplets results in formation of the discrete particles that form the dry powder compositions which are then collected in a separation operation 30. Each of these unit operations is described in greater detail below.

The atomization process 10 may utilize any one of several conventional forms of atomizers. The atomization process increases the surface area of the starting liquid. Due to atomization there is an increase in the surface energy of the liquid, the magnitude of which is directly proportional to the surface area increase. The source of this energy increase depends on the type of atomizer used. Any atomizer (rotary, centrifugal, sonic, pressure, two fluid) which is capable of producing droplets with a mass median diameter of less than about 100 microns, is suitable.

The feedstock for the process may be a solution, suspension, colloidal system, or other dispersion of an active agent in a suitable solvent, or co-solvent system, and is preferably a homogenous solution. The active agent comprises a drug, pharmaceutical, compound, formulation or co-formulation, which is desired to be spray-dried. In one embodiment, the active agent is present as a solution in water. Alcohol/water co-solvent systems according to this invention may also be employed. Other suitable solvents include, but are not limited to, alcohols such as methanol, ketones such as acetone, polar aprotic solvents, hydrogenated hydrocarbons such as methylene chloride, hydrocarbons such as cyclohexane, and mixtures thereof. The total dissolved solids, including the insoluble active agent and other carriers, excipients, etc., that may be present in the final dried particle, may be present at a wide range of concentrations, typically being present at from about 0.1% by weight to about 50% by weight, and often about 1% to about 25% by weight. It will thus be understood that the term “feedstock” as used herein is used broadly and encompasses mixtures such as solutions, slurries, suspensions, emulsions, microemulsions, multiple emulsions, and reverse emulsions.

The drying operation 20 is performed next to evaporate liquid from the droplets produced by the atomization operation 10. In one embodiment, the drying comprises introducing energy to the droplets, typically by mixing the droplets with a heated gas which causes evaporation of the water or other liquid medium. In one embodiment, the mixing is done in a spray dryer or equivalent chamber where a heated gas stream has been introduced. In one embodiment, the heated gas stream may flow concurrently with the atomized liquid; in other embodiments a counter-current flow, cross-current flow, or other flow pattern of the heated gas is employed. It is also possible to perform the drying operation in multiple stages as described, for example, in more detail in WO 01/00312 the disclosure of which is incorporated by reference in its entirety, and in particular with regard to drying apparatus, steps methods and conditions.

The drying rate may be controlled based on a number of variables, including the droplet size distribution, the inlet temperature of the gas stream, the outlet temperature of the gas stream, the inlet temperature of the liquid droplets, and the manner in which the atomized spray and hot drying gas are mixed. In one embodiment, the drying gas stream has an inlet temperature of at least about 70° C., and may be at least about 120° C., at least about 135° C., at least about 145° C., and may often be over about 175° C., or even as high as about 200° C., depending on the active agent being dried. At least in part, the inlet temperature of the heated gas drying stream depends on the lability of the active agent being treated. The outlet temperature is usually in the range of about 50-100° C. The drying gas may be moved through the system using conventional blowers or compressors.

The separation operation 30 is selected to achieve high efficiency collection of the particles produced by the drying operation 20. Any of several conventional separation operations may be used, although in some cases they could be modified to assure collection of a specified particle size range. In one or more embodiments, separation is achieved using a cyclone separator. Other separators, such as filters, for example, a membrane medium (bag filter), a sintered metal fiber filter, or the like may also be used. The separation operation should achieve collection of at least about 70% of all particles, and in some embodiments collects more than about 85%, more than about 90%, or even more than about 95% of such particles.

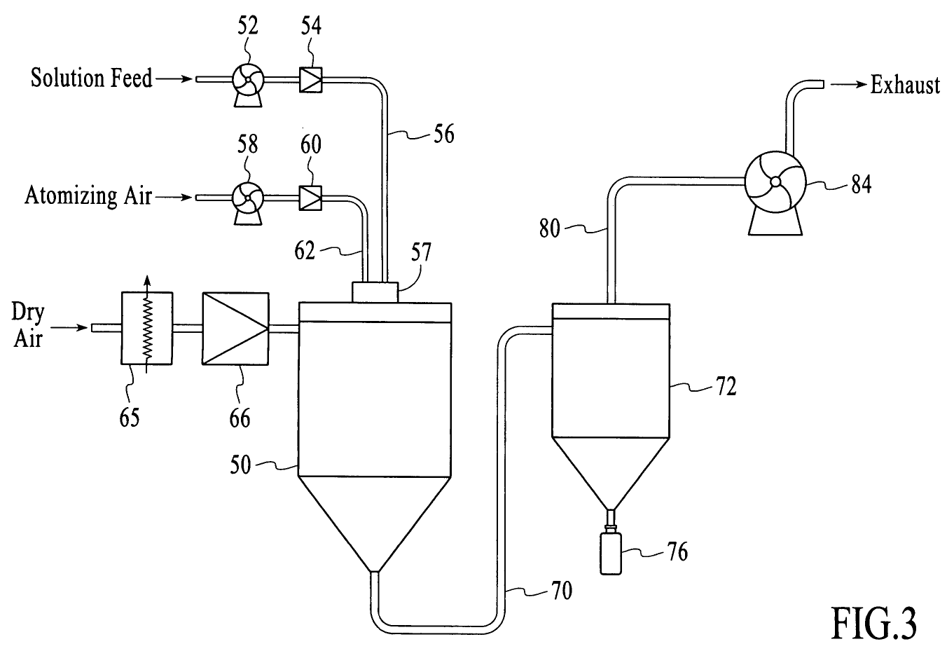

Referring now to FIG. 3, one embodiment of a spray-dryer system is described. The system includes a spray dryer 50, which may be a commercial spray dryer such as those available from suppliers such as Buchi, Niro, APV, Yamato Chemical Company, Okawara Kakoki Company, and others. The spray dryer 50 is provided with a feedstock as described above through a supply pump 52, filter 54, and supply line 56. The supply line 56 is connected to an atomizer 57. Atomizing air is supplied from a compressor 58, a filter 60, and line 62 to the atomizer 57. Drying air is also provided to the spray dryer 50 through a heater 65 and a filter 66.

In this embodiment, dried particles from the spray dryer 50 are carried by the air flow through conduit 70 to a separator 72. In one embodiment, the separator 72 comprises a cyclone. Alternatively, the separator 72 may be a filter, with filter media such as bag filters, cloth filters, and cartridge filters. The dried particles comprising powder are collected in a particle collection canister 76, which may be periodically be removed and replaced. The dry powder in the canister 76 may be used for packaging in unit dosage or other forms. The carrier gas passes out from the top of the separator 72 through line 80 and an exhaust fan 84.

As one alternative to spray drying, the liquid may be removed from the solution, slurry, emulsion, or suspension by other known techniques. For example, the liquid may be removed by freeze drying (lyophilization), vacuum drying, spray freeze drying, evaporation, bubble drying, or the like. In one or more embodiments, spray drying is often advantageous in terms of its efficiency and reproducibility.

Other suitable processes for co-forming losartan and an excipient include hot melt and extrusion processes. For example a feedstock may consist of losartan and excipient mixed together and heated to create a homogeneous hot melt. This feedstock can then be processed using a spray congealing operation to create homogeneous, amorphous particles. Alternatively, the hot melt could be processed through an extrusion operation to yield a granular product.

In other embodiments of the present invention, the non-crystalline formulation may be produced by contacting the liquid containing the losartan with an anti-solvent. For example, in one version, the liquid may comprise one or more organic solvents in which the losartan is dissolved or suspended. The liquid may be contacted by a compressed gas, such as a supercritical or near supercritical anti-solvent gas, to rapidly remove the organic solvent and thereby extract particles comprising losartan. In one particular version, the anti-solvent gas may be supercritical carbon dioxide, for example.

A solvent removal process using a supercritical or near-critical fluid involves contacting a solution or suspension containing losartan in a fluid (the “losartan solution/suspension”) with a compressed fluid (generally a supercritical or near-critical fluid) anti-solvent under conditions which allow the anti-solvent to extract the fluid from the losartan solution/suspension and to cause particles comprising losartan to precipitate from the solution/suspension. The conditions are such that the fluid mixture formed between the anti-solvent and the extracted fluid is still in a compressed (generally supercritical or near-critical) state. The anti-solvent fluid should generally be a nonsolvent for the losartan and be miscible with the fluid. In the context of this or any other solvent removal process, a solution may be construed to include a suspension or dispersion.

In one or more versions, the solvent removal process is a supercritical fluid particle formation process, such as the process known as the “SEDS™” (Solution Enhanced Dispersion by Supercritical fluids) process of Nektar Therapeutics in San Carlos, Calif. and in Bradford, United Kingdom. In one version, this process involves using the anti-solvent fluid substantially simultaneously both to extract the vehicle from, and to disperse, the losartan solution/suspension. In this context, ‘disperse’ refers generally to the transfer of kinetic energy from one fluid to another, usually implying the formation of droplets, or of other analogous fluid elements, of the fluid to which the kinetic energy is transferred. Examples of Nektar Therapeutics' supercritical fluid processes are described in PCT Publications WO 95/01221, WO 96/00610, WO 98/36825, WO 99/44733, WO 99/59710, WO 01/03821, WO 01/15664, WO 02/38127 and WO 03/008082. Other suitable processes are described in PCT Publications WO 99/52507, WO 99/52550, WO 00/30612, WO 00/30613, WO 00/67892 and WO 02/058674. All of these documents are incorporated herein by reference in their entireties. The target solution/suspension and the anti-solvent are preferably contacted with one another in the manner described in WO 95/01221 and/or WO 96/00610, being co-introduced into a particle formation vessel using a fluid inlet which allows the mechanical energy (typically the shearing action) of the anti-solvent flow to facilitate intimate mixing and dispersion of the fluids at the point where they meet. The target solution/suspension and the anti-solvent preferably meet and enter the particle formation vessel at substantially the same point, for instance via separate passages of a multi-passage coaxial nozzle. Alternatively, or additionally, the supercritical fluid process may be of the type described in WO 03/008082, which is incorporated herein by reference in its entirety, in which the target solution/suspension and the anti-solvent enter the vessel at separate, although close, locations.

Reference to an anti-solvent fluid being in a compressed state means that, at the relevant operating temperatures, it is above its vapor pressure, preferably above atmospheric pressure, more preferably from about 50 to 250 bar. The anti-solvent fluid is preferably a fluid which is a gas at atmospheric pressure and ambient temperature. Preferably, “compressed” means close to, at or more preferably above the critical pressure PC for the fluid concerned. The anti-solvent is preferably a supercritical or near-critical fluid or may alternatively be a compressed liquid. A “supercritical fluid” is a fluid at or above its critical pressure (P_{c}) and its critical temperature (T_{c}) simultaneously. A “near-critical fluid” is either (a) above its T_{c} but slightly below its P_{c} or (b) above its P_{c} but slightly below its T_{c} or (c) slightly below both its P_{c} and T. The terms “compressed fluid”, “supercritical fluid” and “near-critical fluid” each encompass a mixture of fluid types, so long as the overall mixture is in the compressed, supercritical or near-critical state respectively.

Various anti-solvents, solvents, and process conditions may be used. The anti-solvent used is preferably supercritical, near-critical or liquid CO₂, especially supercritical CO₂. Preferred solvents include one or more of methanol, ethanol, isopropyl alcohol, acetone, tetrahydrofuran, ethylacetate, dimethylformamide, dichloromethane, MeCN (acetonitrile), N,N-dimethylacetamide (DMA). Hydroxylic solvents are particularly preferred. The processing conditions are preferably chosen to produce particles of desired sizes and/or to reduce residual solvent levels. If losartan is co-formulated with an excipient, and the SCF™ particle precipitation process is used, the excipient is preferably soluble or miscible with the solvent. Excipients with varying degrees of hydrophilicity may thus be suitable depending upon the solvent employed in the SCF™ process.

By “sonic velocity” and “supersonic velocity” is meant respectively that the velocity of the anti-solvent fluid as it enters the vessel is the same as or greater than the velocity of sound in that fluid at that point. By “near-sonic velocity” is meant that the anti-solvent velocity on entry into the vessel is slightly lower than, but close to, the velocity of sound in that fluid at that point—for instance its “Mach number” M (the ratio of its actual speed to the speed of sound) is greater than about 0.8, preferably greater than about 0.9 or about 0.95. Generally speaking, in the method of the invention, the Mach number for the anti-solvent fluid on entering the particle formation vessel may be between about 0.8 and about 1.5, preferably between about 0.9 and about 1.3.

In one or more embodiments, the method of the present invention comprises a method for forming a substance, or co-forming two or more substances, in particulate form, the method comprising introducing into a particle formation vessel (a) a solution or suspension of the target substance in a fluid vehicle (the “target solution/suspension”) and (b) a compressed fluid anti-solvent for the substance, and allowing the anti-solvent fluid to extract the vehicle from the target solution/suspension so as to form particles of the target substance, wherein (i) the pressure in the particle formation vessel is P₁ which is preferably greater than the critical pressure P_{c} of the anti-solvent, (ii) the anti-solvent is introduced through a restricted inlet so as to have a back pressure of P₂, where P₂ is greater than P₁, (iii) the temperature in the particle formation vessel is T₁ which is preferably greater than the critical temperature T_{c} of the anti-solvent, (iv) the anti-solvent is introduced into the vessel at a temperature T₂, where T₂ is greater than T₁, (v) T₁ and T₂ are such that Joule-Thomson cooling of the anti-solvent as it enters the vessel does not reduce the anti-solvent temperature to below that required of it at the point of particle formation (and are preferably such that the anti-solvent temperature does not fall below T_{c} within the vessel) and (vi) P₁, P₂, T₁ and T₂ are such that the anti-solvent fluid has a sonic, near-sonic or supersonic velocity as it enters the particle formation vessel.

Although not intending to be bound by theory, it is believed that in the method of the invention, a so-called “Mach disk” is generated in the anti-solvent flow downstream of the second fluid inlet means. In this region the fluid velocity will change abruptly to sub-sonic thus generating shock waves in the fluids present (in effect a continuous, low volume, supersonic boom). These shock waves are thought to aid mixing and dispersion of the target solution/suspension with the anti-solvent. Moreover they will propagate in the direction of the anti-solvent flow, rather than in a counter-current sense.

The arrangement of the first and second inlet means will preferably be such that the Mach disk is generated upstream (in the direction of anti-solvent flow) of the point of entry of the target solution/suspension into the particle formation vessel. It should occur in line with the longitudinal axis of the second inlet means, i.e., in line with the direction of anti-solvent flow.

The near-sonic, sonic or supersonic anti-solvent velocity is ideally achieved, in one or more methods of the present invention, by the use of appropriate anti-solvent flow rates, back pressures and/or operating temperatures, and preferably without the aid of mechanical, electrical and/or magnetic input such as for example from impellers, impinging surfaces especially within the anti-solvent introducing means, electrical transducers and the like. Introducing the anti-solvent via a convergent nozzle, ideally as a single fluid stream, may also help in the achievement of appropriate fluid velocities.,

The use of near-sonic, sonic or supersonic anti-solvent velocities can allow achievement of smaller particle sizes and narrower size distributions in GAS-based particle formation processes. In particular it can allow the formation of small micro- or even nano-particles, for instance of volume mean diameter less than about 5 microns, preferably less than 2 microns, more preferably less than about 1 micron. Such particulate products preferably have narrow size distributions, such as with a standard deviation of 2.5 or less, more preferably 2.0 or less, most preferably 1.9 or even 1.8 or less.

The use of near-sonic, sonic or supersonic anti-solvent velocities also appears to lead to more efficient vehicle extraction, thus potentially yielding particles with lower residual solvent levels, less agglomeration and generally improved handling properties.

Preferably the two fluids meet immediately downstream of the point of anti-solvent entry. “Immediately” in this context implies a sufficiently small time interval (between the anti-solvent entering the particle formation vessel and its contact with the target solution/suspension) as preferably still to allow transfer of mechanical energy from the anti-solvent to the solution/suspension so as to achieve dispersion. Nevertheless, there is still preferably a short interval of time between anti-solvent entry and fluid contact so as to eliminate, or substantially eliminate or at least reduce, the risk of apparatus blockage due to particle formation at the point of anti-solvent entry. The timing of the fluid contact will depend on the natures of the fluids, the target substance and the desired end product, as well as on the size and geometry of the particle formation vessel and the apparatus used to introduce the fluids and on the fluid flow rates. The contact may occur within about 0.001 to about 50 milliseconds, or within about 0.001 to abut 25 milliseconds. The contact preferably occurs within about 0.001 to about 20 milliseconds, such as within about 0.01 to about 10 milliseconds, of the anti-solvent entering the particle formation vessel.

At the point where the target solution/suspension and the anti-solvent meet, the angle between their axes of flow may be from about 0 degrees (i.e., the two fluids are flowing in parallel directions) to about 180 degrees (i.e., oppositely-directed flows). In one embodiment of the present invention, they meet at a point where they are flowing in approximately perpendicular directions, i.e., the angle between their axes of flow is from about 70 to about 110 degrees, more preferably from about 80 to about 100 degrees, such as about 90 degrees. In another one embodiment of the present invention, the flows of target solution/suspension and the anti-solvent meet at a point where they are flowing in approximately parallel directions, i.e., the angle between their axes of flow is from about 0 to about 70 degrees, more preferably from about 0 to about 30 degrees, such as about 0 degrees.

When carrying out one or more embodiments of the present invention, the particle formation vessel temperature and pressure may be controlled so as to allow particle formation to occur at or substantially at the point where the target solution/suspension meets the anti-solvent fluid. The conditions in the vessel must generally be such that the anti-solvent fluid, and the solution which is formed when it extracts the vehicle, both remain in the compressed (preferably supercritical or near-critical, more preferably supercritical) form whilst in the vessel. For the supercritical, near-critical or compressed solution, this means that at least one of its constituent fluids (usually the anti-solvent fluid, which in general will be the major constituent of the mixture) should be in a compressed state at the time of particle formation. There should at that time be a single-phase mixture of the vehicle and the anti-solvent fluid, otherwise the particulate product might be distributed between two or more fluid phases, in some of which it might be able to redissolve. This is why the anti-solvent fluid needs to be miscible or substantially miscible with the vehicle.

The flow rate of the anti-solvent fluid relative to that of the target solution/suspension, and its pressure and temperature, should be sufficient to allow it to accommodate the vehicle, so that it can extract the vehicle and hence cause particle formation. The anti-solvent flow rate will generally be higher than that of the target solution/suspension--typically, the ratio of the target solution/suspension flow rate to the anti-solvent flow rate (both measured at or immediately prior to the two fluids coming into contact with one another) will be about 0.001 or greater, preferably from about 0.01 to about 0.2, more preferably from about 0.03 to about 0.1. The anti-solvent flow rate will also generally be chosen to ensure an excess of the anti-solvent over the vehicle when the fluids come into contact, to minimize the risk of the vehicle re-dissolving and/or agglomerating the particles formed.

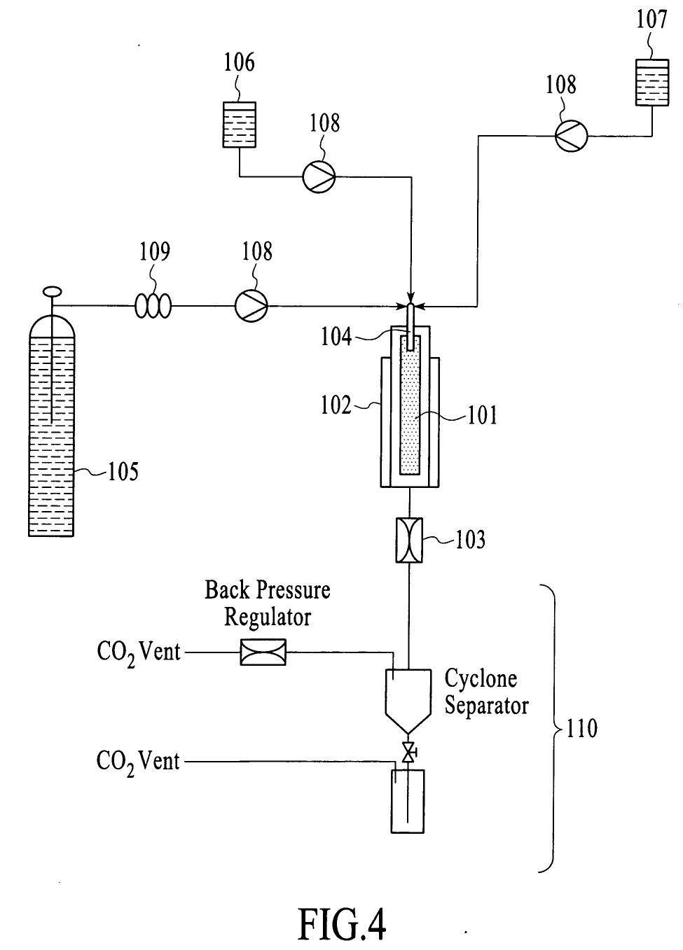

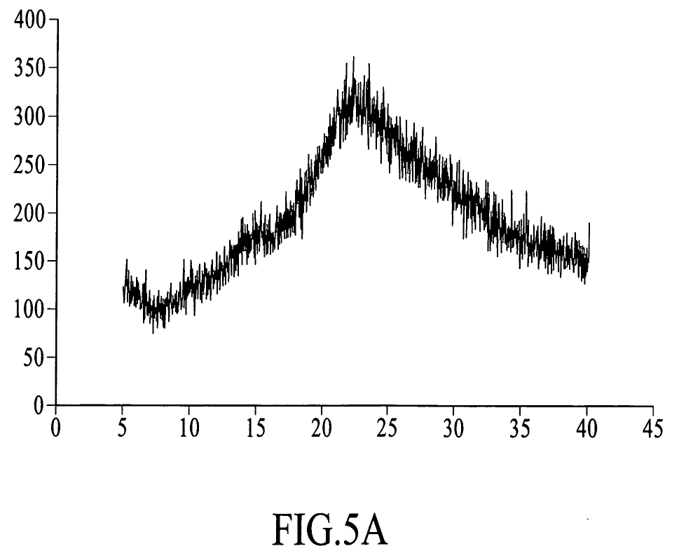

FIG. 4 shows one embodiment of an apparatus suitable for carrying out methods in accordance with the present invention. Reference numeral 100 denotes a particle formation vessel, within which the temperature and pressure can be controlled by means of a heating jacket 102 and back a pressure regulator 103. The vessel 100 contains a particle collection device (not shown) such as a filter, filter basket or filter bag. A fluid inlet assembly 104 allows introduction of a compressed (typically supercritical or near-critical) fluid anti-solvent from source 105 and one or more target solutions/suspensions from sources such as 106 and 107. The elements labeled 108 are pumps, and 109 is a cooler. A recycling system 110 allows solvent recovery.

The fluid inlet assembly 104 may for example take the forms shown in U.S. Pat. No. 6,063,138 and/or U.S. Pat. No. 5,851,435, the disclosures of which are incorporated by reference in their entireties, and in particular with regard to apparatus, steps, methods and conditions. The fluid inlet assembly 104 includes a nozzle (not shown) for introduction of the anti-solvent fluid. The nozzle may comprise a single passage of circular cross section, with a circular outlet, or may alternatively comprise a multi-component nozzle, with anti-solvent introduced through one or more of its passages and the remaining passages either closed off or else used to introduce additional reagents. (For example, a multi-passage nozzle of the type described in WO-95/01221 and/or corresponding U.S. Pat. No. 5,851,453 or WO-96/00610 may be used). Such nozzles have two or more concentric (coaxial) passages, the outlets of which are typically separated by a short distance to allow a small degree of internal mixing to take place between fluids introduced through the respective passages before they exit the nozzle. The anti-solvent could for instance be introduced through the inner passage of such a nozzle, traversing a small “mixing” zone as it exits that inner passage and then passing through the main nozzle outlet into the particle formation vessel).

The opening at the outlet end (tip) of the nozzle may have a diameter in the range of about 0.05 to about 2 mm, preferably between about 0.1 and about 0.3 mm, typically about 0.2 mm. The outlet end of the nozzle may be tapered depending upon the desired velocity of the fluids introduced through the nozzle; an increase in the angle may be used, for instance, to increase the velocity of the supercritical fluid introduced through the nozzle and hence to increase the amount of physical contact between the supercritical fluid and the vehicle.

A pure non-crystalline losartan potassium formulation tends to be physically unstable. Accordingly, in one or more versions of the present invention, a non-crystalline formulation comprising losartan is formulated so as to improve its physical stability. For example, the improved stability may be provided by combining the non-crystalline losartan with a stabilizing excipient. The stabilizing excipient is provided in a sufficient quantity to reduce the tendency of the non-crystalline losartan to convert to a crystalline form. The losartan and a stabilizing excipient may be formulated together by conventional methods such as blending the two ingredients together. Preferably, the stabilizing excipient is in intimate contact with the non-crystalline losartan. The stabilizing excipient may be either non-crystalline or crystalline, as long as it serves to maintain the losartan in a non-crystalline form.

In one or more versions, the formulation is made up of particles, and the particles comprise non-crystalline losartan and an excipient, i.e. both the losartan and the stabilizing excipient are present in the same formulated particle. By providing the stabilizing excipient and the losartan in the same particle, the excipient and the losartan are in greater contact and the stabilizing excipient is better able to assert its stabilizing influence on the losartan. In one or more versions, the losartan and the excipient are formulated so that there is provided a solid dispersion of one component in another, such as an intimate mixture of losartan dispersed in a matrix of the stabilizing excipient, or a solid solution of the components, whereby an intimate association results. In one or more versions, the particles comprising non-crystalline losartan and excipient may be formulated by adding the excipient to the liquid in the product methods described above. For example, losartan and a stabilizing excipient may be dissolved or suspended in an aqueous or organic solvent and the particles may be formed by removing the solvent by spray drying, freeze drying, spray freeze drying, evaporation, supercritical fluid extraction, or other solvent removal technique.

The stabilizing excipient may be any excipient that serves to reduce the conversion of non-crystalline losartan to crystalline losartan when compared to non-crystalline losartan in the absence of the stabilizing excipient. For example, the excipient may comprise one or more polymeric or oligomeric excipients, such as polyvinyl acetate (PVA), vinylpyrrolidone/vinyl acetate copolymer (PVP-VA), vinylpyrrolidone/vinyl acetate copolymer in a VP:VA of 60:40 (PVP-VA 64), poly ethylene oxide (PEO), cellulose, starch, polyethylene glycol (PEG), hydroxypropyl cellulose (HPC), hydroxyl propyl methyl cellulose (HPMC), and their copolymers and derivatives; carbohydrates; polyols; sugars; oligo saccharides such as cyclodextrins; proteins, peptides and amino acids; lipids and modified lipids such as lipid-PEG and lipid-sugar esters; salts; citric acid; citrates; known glass formers; or the like. Polyvinylpyrrolidone may be a suitable stabilizing excipient under certain processing conditions, such as under SEDS™ processing, however, it has been found that polyvinylpyrrolidone as the sole excipient, when processed under certain spray-drying conditions, produces a powder which is difficult to handle and process into a tablet, and is additionally not sufficiently stable. Some stabilizing excipients are described in U.S. Pat. No. 6,582,728, and in PCT WO 01/15664, the entire disclosures of which are incorporated herein by reference in their entireties, and in particular those portions relating to excipients.

Examples of other polymeric or oligomeric excipients for formulation with losartan according to the invention include other celluloses and cellulose derivatives, such as alkyl (for example, methyl or ethyl) cellulose, hydroxyalkyl celluloses (such as hydroxypropylmethyl cellulose, hydroxypropylmethyl cellulose phthalate, hydroxyethyl cellulose, hydroxypropyl cellulose), carboxymethylcelluose, sodium carboxymethyl cellulose, microcrystalline cellulose, microfine cellulose) or mixtures thereof; traditional “natural” source materials, their derivatives and their synthetic analogues, such as acacia, tragacanth, alginates (for instance calcium alginate), alginic acid, starch, agar, carrageenan, xanthan gum, chitosan, gelatin, guar gum, pectin, amylase or lecithin; homo- and co-polymers of hydroxy acids such as lactic and glycolic acids; hydrated silicas, such as bentonite or magnesium aluminium silicate; polymeric surfactants, such as polyoxyethylene or polyoxypropylene, or polyalkylene oxides such as polyethylene oxides;phospholipids, such as DMPC (dimyristoyl phosphatidyl choline), DMPG (dimyristoyl phosphatidyl glycerol) or DSPC (distearyl phosphatidyl choline); carbohydrates, such as lactose, sucrose, dextrans, cyclodextrins or cyclodextrin derivatives; mannitol; dendrimeric polymers, such as those based on 3,5 hydroxy benzyl alcohol;poly(ε-caprolactones), DL-lactide-co-caprolactones and their derivatives;poly(orthoester)s and poly(orthoester)/poly(ethylene glycol) copolymers, including block copolymers, such as are described in U.S. Pat. No. 5,968,543 and U.S. Pat. No. 5,939,453, the entire disclosures of which are incorporated herein by reference in their entireties, and in particular those portions relating to polymers and/or excipients. Derivatives of such polymers, such as polymers with incorporated esters of short chain a-hydroxy acids or glycolic-co-lactic acid copolymers; or mixtures thereof, are additionally suitable.

Preferred excipients, especially when the liquid removal process comprises spray-drying, are those which have a T_{g} of above about 40° C., and preferably above about 50° C. In some versions, the T_{g} may be above about 55 or 60 or 65 or 70° C. Particularly preferred excipients, especially when the liquid removal process comprises spray-drying, are those which, when formulated or co-formulated with the losartan in accordance with one or more embodiments of the present invention herein, result in a formulation or co-formulation T_{g} of above about 40° C., and preferably above about 50° C. In some versions, the formulation T_{g} may be above about 55 or 60 or 65 or 70° C. In one or more embodiments copolymers are preferred excipients. Such copolymers my comprise block, alternating, random, graft, branched, substituted and combinations thereof. Copolymers of vinyl pyrrolidone with vinyl acetate and/or vinyl alcohol are particularly preferred. It is additionally preferred that a ratio of vinyl pyrrolidone:vinyl acetate be about 60:40, or in ratios such as about 80:20, 70:30, 50:50, 30:70, 40:60 and 20:80.

In one or more versions of the formulation according to the invention, an oligomeric or polymeric stabilizing excipient is present in an amount by weight sufficient, following formulation with losartan, to provide improved stability to the non-crystalline losartan. In one or more embodiments, the improved stability comprises physical stability which is at least comparable to that attained by a crystalline form of losartan. In one or more embodiments, the improved stability comprises chemical stability which is at least comparable to that attained by a crystalline form of losartan. In other embodiments, the improved stability comprises a formulation which maintains its non-crystalline form when stored at 25° C. and 60% relative humidity for a period of at least one week, preferably at least one month, more preferably at least three months. In some embodiments, the formulation maintains its non-crystalline form when stored at 25° C. and 60% relative humidity for a period of at least about one year. In other embodiments, the improved stability comprises a formulation which maintains its non-crystalline form when stored at 40° C. and 75% relative humidity for a period of at least one week, more preferably at least one month, more preferably at least three months.

Generally, in terms of weight percentage, the excipient is present at a concentration in the range of from 1 to 99.9% w/w, preferably from 5% to7O%, more preferably from 10% to 50% w/w of the formulation. The losartan may be present in the complementary (to the excipient) amount, and in one or more versions is present in an amount of between about 0.1 to 99.9% by weight, and often is present from about 1 to 50%, typically from about 5 to 25% by weight.

The formulation according to the invention is preferably in particulate form, especially in the form of fine particles having a volume mean diameter (VMD) of about 5 to about 200 μm preferably about 10 μm to about 100 μm more preferably from about 10 μm to about 50 μm, or about 15 μm to about 30 μm. In some embodiments, particle sizes are about 20 or 22 μm, or in a range thereof. Particle sizes may be measured for instance using a laser diffraction sensor such as the Helos™ system available from Sympatec GmbH, Germany (which provides a geometric projection equivalent (mass mean diameter, MMD)). Volume mean diameters may be obtained using commercially available software packages.

Following formulation with at least one excipient, the losartan will have improved physical stability with respect to reversion to crystalline form, for at least one week, more preferably at least one month, and most preferably at least three months. By “stable” is meant that over the specified time period, there is no significant change in the X-ray diffraction (XRD) pattern of the formulation and, where measurable, in its differential scanning calorimetry (DSC) profile. Preferably there is no significant change in the dissolution profile of the losartan formulation over time. Preferably there is little or no (for example less than about 10%, preferably less than about 5%, more preferably less than about 1%) change in degree of crystallinity of the losartan within the formulation with respect to the initial amount. Particularly preferably, there is no detectable crystalline losartan present in the formulation either before or after storage. Stability may be assessed by storing the formulation according to the invention at ambient temperature, for example from about 18 to about 25° C., or from about 20 to about 23° C., such as about 22° C., or at the accepted industrial standard temperature of about 25 ° C., and at up to about 20% or 30% or 40% or 60% or even 75% relative humidity (RH). In one particular assessment, the temperature is about 25° C. and the relative humidity is about 60%. Higher storage temperatures and/or humidity conditions may be used, in conventional manner, to establish shelf life for longer term storage under ambient conditions. Conventional thermal cycling procedures such as freeze/thaw cycling, may be employed in some circumstances, for example, stability assessment of non-solid formulations. For example, an accelerated storage assessment may be performed at about 40° C. and about 75% relative humidity. The formulation according to the invention is preferably stable, for the periods mentioned above, when stored at about 25° C. and up to about 60% RH for a period of at least one year, more preferably at least eighteen months, and most preferably at least twenty-four months. Even more preferably, the formulation is considered stable when stored at about 40° C., most preferably at about 40° C. and up to about 75% RH for a period of at least one year, more preferably at least eighteen months, and most preferably at least twenty-four months. As a general guide, a formulation tested as stable under accelerated storage conditions for three months will be stable under ambient storage conditions for at least about two years.

The degree of crystallinity of the formulation may be assessed by conventional techniques, for example using X-ray powder diffraction (XRPD) techniques, particularly high resolution X-ray powder diffraction using a synchrotron radiation source. Levels of non-crystalline or amorphous phase may also be assessed by reference to its moisture uptake at any given temperature and humidity.

Bioavailability may be assessed, according to standard procedures, with reference to the release profile of the active substance, with time, into the patient's bloodstream. It may be measured for example as either the maximum plasma concentration of active achieved following administration (Cₘₐₓ), or as the area under the plasma concentration curve (AUC) integrated from time zero (the point of administration) to a suitable endpoint or to infinity. Bioavailabilty can also be estimated using standard dissolution rate tests.

The formulations according to one or more embodiments of the present invention may be further formulated into a pharmaceutical composition. A pharmaceutical composition according to the invention may take the form of any delivery form conventional in the art. The composition may take the form of a solid composition such as a powder, granulate or tablet, for example, or a liquid form such as a solution or suspension (including more viscous forms such as pastes and gels) suitable for oral delivery. Alternatively, pharmaceutical compositions according to the invention may be presented in a form suitable for topical application (for instance as a gel or paste), as a solution or suspension for injection or as a suppository.

Pharmaceutical compositions according to one or more embodiments of the invention may comprise additional active substances and/or excipients, which may or may not be included along with the losartan and the excipient as part of the formulation of the invention. For example, the pharmaceutical composition(s) may comprise the losartan formulation of the present invention plus a diuretic, such as hydrochlorothiazide in its commercially available form, that is added to the composition. Alternatively, the hydrochlorothiazide or other active agent may be formulated to be in the same particle as the losartan by adding the hydrochlorothiazide to the liquid containing the losartan during the processing of the losartan. The liquid is then removed from the solution of losartan and hydrochlorothiazide or the solution of losartan, hydrochlorothiazide and excipient, by spray-drying, supercritical processing or any other solvent removal process as described herein to result in particles of the desired characteristics. In one or more embodiments, the formulation comprising losartan, hydrochlorothiazide and excipient may provide a bio equivalent substantially equal to that of a commercially available product, such as HYZAAR® tablets. The hydrochlorothiazide may also be dry-blended in with the tablet formulation. The pharmaceutical compositions according to the invention may include other additives such as those typically used in pharmaceutical dosage formulations, for instance flavorings and sweeteners, colors, bulking agents, tablet lubricants and disintegrating agents.

The non-crystalline form of losartan may be formed by adding the losartan to a liquid and removing the liquid in a manner that produces particles comprising non-crystalline losartan, such as by using one or more of the solvent removal or solid extraction techniques discussed above. In one or more versions, a crystalline form of losartan may be used as the starting material that is added to the liquid. The crystalline losartan potassium, for example, is dissolved in the solvent and the solvent is removed by a process that produces the non-crystalline losartan. Alternatively, the steps of producing crystalline losartan and then using the crystalline losartan as a starting material can be avoided. The free acid of losartan can be added to a substantially equal mole of a compound containing an alkali earth metal or alkaline earth metal and a counter ion in a water or other solution. For example, the alkali earth metal or alkaline earth metal may comprise one or more of Li, Na, K, Rb, Cs, Fr, Be, Mg, Sr and Ba, and the counter ion may comprise one or more of chloride, bromide, iodine, carbonate, sulfide, and hydroxide. In one or more versions, the losartan free acid and KOH are added to a water solution. The losartan free acid and the alkali and/or alkaline earth metal react to form a losartan salt, such as losartan potassium. This solution, which now contains a losartan salt, may then be the liquid or part of the liquid that is processed to produce the non-crystalline losartan. In other versions, a liquid is provided which has losartan free acid in solution, such as a mother liquor from a losartan synthesis process. To this solution an equal mole of the salt, as described above, may be added and then the liquid of this solution may be removed to produce the non-crystalline losartan salt. When a process is being performed that uses a nonaqueous organic solution, such as a SEDS™ process, it is often desirable to use potassium methoxide (KOMe) instead of KOH. When an excipient is to be included in the produced particles, the excipient may be added to the solution containing the losartan free acid and the potassium or other alkali earth metal or alkaline earth metal, plus counter ion. In another version, a pure non-crystalline losartan may be produced using any of the techniques described herein and the pure non-crystalline losartan may be used as the starting material for making particles comprising non-crystalline losartan and a stabilizing excipient according to any of the techniques described herein.

Tablet Dosage Form

A non-crystalline form of losartan may be made by spray-drying a solution of losartan potassium and PVP-VA, in accordance with one or more embodiments of the present invention. The spray-dried powder may then be formulated, with additional excipients, into an appropriately-sized tablet dosage form, for example, containing 100 mg of losartan per tablet. A dry granulation process, such as roller compaction, may be used to make the granules. Alternatively or additionally, a wet granulation process as known in the art, may be used to make the granules. In either case, the granules can than be compressed into tablets, also by means as known in the art. In one or more embodiments, a tablet dissolution profile is preferably comparable to (at least parity or near parity with) a commercially-available dosage form, especially 100 mg COZAAR®. In other embodiments, a tablet dissolution profile is preferably better than commercially-available dosage form, especially 100 mg COZAAR®. The tablet formulations may be made as described herein to be preferably chemically and physically stable for at least one year, preferably two years at room temperature, and/or preferably stable for at least one year under accelerated storage conditions. The formulations additionally may be scaled to production-sized batches.

In one or more versions, a table formulation is made wherein the tablet contains no binder or disintegrant, or both, and is preferably slowly eroding and/or disintegrating, such as not breaking apart rapidly in water. In one or more embodiments, a tablet formulation which contains no binder or disintegrant, or both provides a desired dissolution rate and/or profile. In other versions, the tablet may further comprise a basifying agent, for example dicalcium phosphate or calcium oxide, to obtain faster dissolution.

The following examples illustrate the formation of non-crystalline and/or stable versions of a formulation comprising losartan. These examples are not intended to limit the scope of the invention.

### EXAMPLE 1

In a first example, a spray drying process is used to produce particles comprising non-crystalline losartan and a stabilizing excipient. In this version, the stabilizing excipient can be any excipient that increases the physical stability of the non-crystalline losartan potassium when compared to a formulation of non-crystalline losartan potassium substantially without the excipient. In one version, the stabilizing excipient comprises a co-polymer, such as a vinyl pyrrolidone vinyl acetate (PVP-VA) co-polymer.

Specifically, the non-crystalline losartan potassium and excipient of Example 1 can be made by performing the following steps:

1. Starting with the commercially available crystalline losartan potassium, the salt is dissolved in water at 0.1 to 20%, preferably at 5-15% solids content.

2. The PVP-VA excipient is then added to the solution in a weight ratio of PVP-VA to losartan potassium of about 1:1.

3. The solution of step 2 is spray-dried, under conditions appropriate to form a free-flowing non-crystalline powder comprising particles of losartan potassium and PVP-VA.

The weight ratio of PVPVA or other stabilizing excipient to losartan potassium comprises from about 0.1:10 to 10:0. 1, preferably from about 1:10 to 10:1, and more preferably about 1:1. The solvent of this example can be removed by other aqueous solvent removal processes, such as evaporation, freeze-drying, spray-freeze drying, bubble drying or vacuum drying. The solvent of this example may alternatively or additionally comprise solvents other than water. For example, ethanol, isopropanol, methanol, other short chain alcohols, esters, ethers, and other low boiling point solvents, and mixtures thereof, may be used.

In one or more versions, the PVP-VA may be replaced by or supplemented with another stabilizing excipient. The stabilizing excipient may be selected to be any excipient that increases the physical stability of the non-crystalline losartan potassium when compared to a formulation of non-crystalline losartan potassium substantially absent the excipient. This increase in physical stability may in terms of the formulations storage life before crystallization and/or may be in terms of its glass transition temperature at a particular relative humidity and/or other physical stability determinants. In one or more versions, the stabilizing excipient is selected that has a higher glass transition temperature than the non-crystalline losartan. In other versions, the stabilizing excipient may be selected so that it has a lower hygroscopicity than the non-crystalline losartan potassium. In other versions, the stabilizing excipient is selected to have both a higher glass transition temperature, and a lower hygroscopicity than the non-crystalline losartan. Examples of stabilizing-effective excipients comprise one or more of: PVP-VA, PVP-VA at different VP:VA ratios, for example VP:VA 60:40 and VP:VA 20:80; CaCl₂, Arginine, Tris, sodium citrate and citric acid, HPMC, ethyl cellulose and mixtures therof. Sugars and sugar polymers can also very effective as a stabilizer against crystallization.

PVP-VA, such as PVP-VA 64, has been determined to be particularly advantageous. PVP-VA is very non-hygroscopic, and the glass transition temperature of PVP-VA remains relatively high (about 50° C.) after exposure to ambient conditions because of this relatively low hygroscopic nature. In addition, PVP-VA is relatively nonsticky which allows for easier tablet formulation processing.

### EXAMPLE 2

Example 2 represents a specific version of Example 1. In the production of Example 2, the following steps were carried out under ambient conditions:

1. 5 g PVPVA 64 was slowly added and dissolved in 90g water under constant stirring at about 60 RPM.

2. 5 g losartan potassium was added into the solution made from step 1, and dissolved using an energy input, such as agitation, as by stirring or sonication. In preferred embodiments, agitation comprises constant stirring at about 60 RPM. The order of steps I and 2 may be reversed.

3. The resultant solution was spray dried into powders by introducing the solution into a spray-dryer, such as a Buchi model 190 mini spray-drier, under conditions to make a free-flowing amorphous powder. In one or more embodiments, such conditions comprise setting the feed rate at 5- 1 0 ml/min and inlet gas temperature at 100-120° C. to provide a relatively quick drying process. In one or more embodiments, it is preferred that the spray-drying step occur soon after preparation of the losartan/PVP-VA solution in order to minimize possible chemical degradation, and more preferably the spay-drying commences immediately. When using a larger spray dryer, the conditions may be adjusted accordingly wherein in one or-more embodiments, a free-flowing powder is obtained with a residual moisture level of about 3-5%, and a T_{g} of above about 40° C. In general, it has been found that a T_{g} above about 40° C. results in a powder which is easy to handle, and easy to process into a tablet.

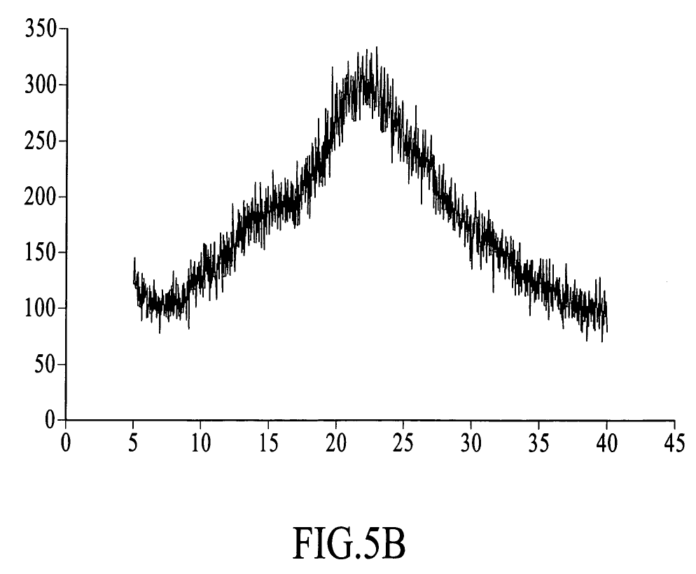

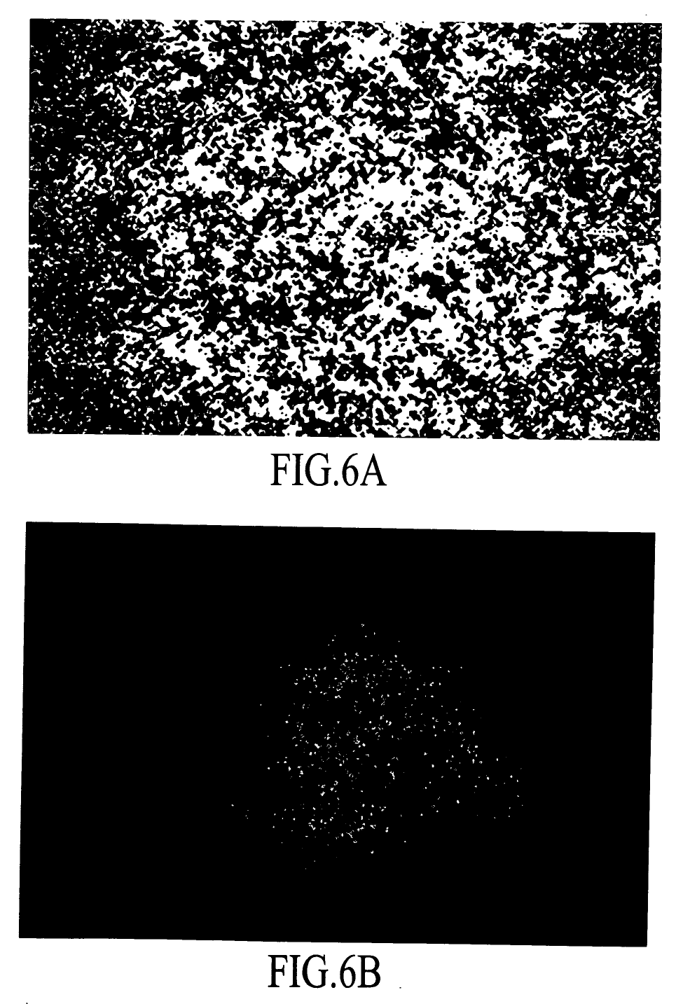

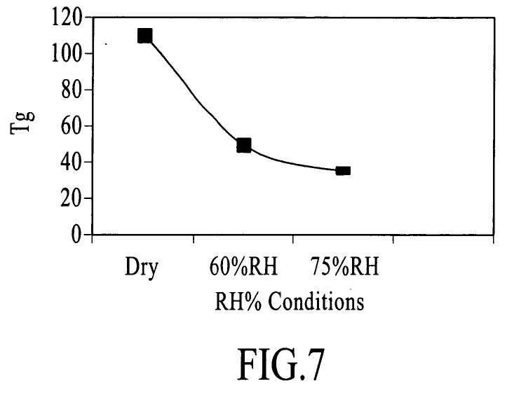

The particles comprising non-crystalline losartan potassium and stabilizing excipient made in accordance with Example 2 have been analyzed and have been found to be non-crystalline with improved physical stability. An X-ray powder diffraction pattern of the powder particles is shown in FIG. 5A. The X-ray pattern shows the powder to be non-crystalline in that no crystallinity-indicative peaks are present. The powder particles were then stored for 1 week at 75% relative humidity at 40° C. After this storage, the particles were X-ray again and the X-ray powder diffraction pattern is shown in FIG. 5B. As can be seen, there is no indication of the conversion of the non-crystalline form to a crystalline form. In addition, polarized light micrographs were taken soon after formulation, FIG. 6A, and after the one week storage described above, FIG. 6B. No crystallization was observed. To further illustrate the improvement in stability over pure non-crystalline forms of losartan, FIG. 7 shows a graph of the glass transition temperature of the particles as a function of relative humidity at 40° C. From FIG. 7, it can be seen that one result of one or more embodiments of the invention is a desirably high glass transition temperature of the spray-dried formulation comprising losartan and an appropriate stabilizing excipient. The particles of Example 2 were also stored for three months at room conditions, and no crystallinity was observed, thus confirming stability.

The particles made by Example 2 were further determined to be advantageous over pure non-crystalline forms. The powder of non-crystalline losartan and PVP-VA formulation remains flowable after exposure to ambient conditions, while the pure non-crystalline losartan powder sticks and agglomerates. Accordingly, the non-crystalline losartan and PVP-VA containing powder formulation has an improved flowability for downstream process such as tablet formation.

### EXAMPLE 3

Example 3 represents another specific version of Example 1, but with the addition of an additional excipient. In the production of Example 3, the following steps are carried out under ambient conditions:

1. 5 g PVPVA 64 was slowly added and dissolved in 90 g water under constant stirring at about 60 RPM.

2. 5 g losartan potassium was added into the solution made from step 1, and dissolved under constant stirring at about 60 RPM.

3. 0.1 g Tris was added into the solution made from step 2, and dissolved under constant stirring at about 60 RPM. Steps 1, 2 and 3 may be performed in any order.

4. The resultant solution is spray dried into a powder form by introducing the solution into a 190 mini-spray-dryer, under conditions to make the amorphous powder. In one embodiment, such conditions comprise setting the feeding rate at about 5-10 ml/min and inlet gas temperature at about 100-120° C. to provide a relatively quick drying process. When using a larger spray dryer, the conditions may be adjusted accordingly wherein in one or more embodiments a free-flowing powder is obtained with a residual moisture level of about 3-5%, and a T_{g} of above about 40° C.

In this version, Tris is added as an additional excipient. The role of the Tris is to serve as a buffer and/or as an additional stabilizing agent. Alternatively or additionally, other buffering agents could be used. Alternatively or additionally, other agents, such as anti-oxidants can be introduced, such as vitamins such as vitamin C and/or vitamin E, methionine, lipoic acid, and the like. Other additional agents, such surfactants and zein (a maize protein) may be added, to form a solution or a suspension. One of the reasons for doing so may be to tailor the properties of the powder, such as processibility and/or stickiness when exposed to humid environments, and dissolution rate when reconstituted into a solution.

The particle produced in accordance with Example 3 have been tested and analyzed. After being exposed to 75% relative humidity at 40° C. for 1 month, analysis revealed no crystallization, thus confirming stability.

### EXAMPLE 4

In a fourth example, a supercritical fluid is used to produce non-crystalline losartan by removing the solvent, such as an organic solvent, from a solution of losartan and a stabilizing excipient. In this version, the stabilizing excipient can be any excipient that increases the physical stability of the non-crystalline losartan potassium when compared to a formulation of non-crystalline losartan potassium substantially absent the excipient.

Specifically, the non-crystalline losartan potassium and excipient of Example 4 can be made by performing the following steps:

1. Starting with the commercially available crystalline losartan potassium, the salt is dissolved in an organic solvent, such as methanol and optionally acetone at 1-20%, with preferably at 2.5-10% solid content.

2. The stabilizing excipient is then added to the solution in a weight ratio of stabilizing excipient to losartan potassium of from about 0.1:10 to about 10:0.1, preferably from about 1:10 to about 10:1, more preferably from about from about 1:2 to about 2:1, and most preferably about 1:1.

3. The solution is contacted with a supercritical fluid or near critical fluid anti-solvent which removes the liquid from the solution of losartan potassium and stabilizing excipient, resulting in a free-flowing powder.

The solvent of this example can be removed by other orgainc solvent removal processed, such as evaporation, freeze-drying, spray-freeze drying, bubble drying or vacuum drying. The solvent of this example may alternatively or additionally comprise other organic solvents. For example, for the SEDS™ process, the desired solutes are dissolved or dispersed in a solvent and or solvent mixture which is miscible with carbon dioxide. Solvent choice comprises, for example, one or more of methanol, ethanol, propan-2-ol, 1-propanol, 2-methyl-1 propranol, butanol, dimethylsulfoxide, dichloromethane, toluene, hexane, ethyl ether, heptane, chloroform, acetone, ethyl acetate, toluene, acetonitrile, isopropyl acetate, methyl acetate, methylethylketone, methylisobutylketone, tetrahydrofuran, cyclohexane, N,N-dimethylformamide and dimethylacetanilide.

The stabilizing excipient may be selected to be any excipient that increases the physical stability of the non-crystalline losartan potassium when compared to a formulation of non-crystalline losartan potassium substantially absent the excipient. This increase in physical stability may be in terms of the formulations storage life before crystallization and/or may be in terms of its glass transition temperature at a particular relative humidity and/or other physical stability determinants. In one or more versions, the stabilizing excipient is selected that has a higher glass transition temperature than the non-crystalline losartan. In other versions, the stabilizing excipient may be selected so that it has a lower hygroscopicity than the non-crystalline losartan potassium. In other versions, the stabilizing excipient may be selected so that it has both a higher glass transition temperature than that of the non-crystalline losartan and a lower hygroscopicity than that of the non-crystalline losartan (such as losartan potassium). In one or more embodiments, suitable stabilizing excipients comprise PVPVA, ethyl cellulose, Eudragit E, hydroxypropyl cellulose and hydroxypropyl beta cyclodextriri and mixtures of the above. Additional stabilizing excipients include cellulose polymers especially enteric cellulose polymers such as cellulose acetate phthalate, hydroxypropyl methylcellulose phthalate etc.

### EXAMPLE 5

Example 5 represents a specific version of Example 4. In the production of Example 5, the following steps are carried out:

1. 4 g hydroxpropyl beta cyclodextrin and 1 g ethyl cellulose are added slowly to a solution containing 50 mL methanol +50 mL acetone. The excipients are dissolved by sonication and or stirring at about 60 RPM.

2. 5 g crystalline losartan potassium is added to the solution made from step 1, and dissolved by sonication and or stirring at about 60 RPM. The order of steps 1 and 2 may be reversed.

3. The solution is processed using a SED™ process using a nozzle with a 200 μm tip for the CO₂ line and a 125 μm tip for the solution line. The conditions used (at pilot plant scale) were reactor vessel pressure of 85 bar, reactor vessel temperature of 40° C., anti solvent (CO₂) flow rate of 12-12.5 kg·hr⁻¹ and a solution flow of 4 ml·min⁻¹. In one or more embodiments, the result is a free flowing powder with a wet T_{g} of about 50-80° C.

In this version, the stabilizing excipient comprises hydroxypropyl beta cyclodextrin in combination with ethyl cellulose, at a weight ratio of excipient mixture to losartan potassium in the range of from 0.10:10 to 10:0.10, more preferably from 1:10 to 10:1, and most preferably 1:1. This mixture of hydroxypropyl beta cyclodextrin with ethyl cellulose is advantageous in that it is particularly effective in stabilizing the non-crystalline losartan. In addition, hydroxypropyl beta cyclodextrin is relatively hygroscopic while ethyl cellulose is not very hygroscopic, therefore a mixture of ethyl cellulose with hydroxpropyl beta cyclodextrin reduces the water uptake of the cyclodextrin molecule. Furthermore, the mixture of hydroxypropyl beta cyclodextrin with ethyl cellulose is relatively non-sticky, i.e. it is not a strong binder. The weight ratio of hydroxypropyl beta cyclodextrin to ethyl cellulose in the mixture may be from 19:1 to 1:4, more preferably from 10:1 to 1:1, most preferably about 4:1.

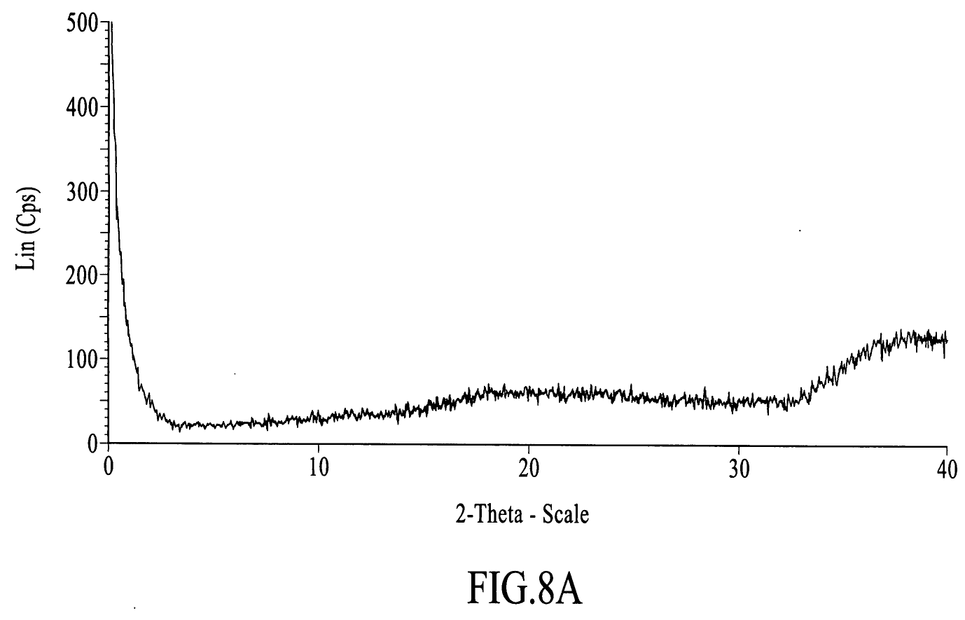

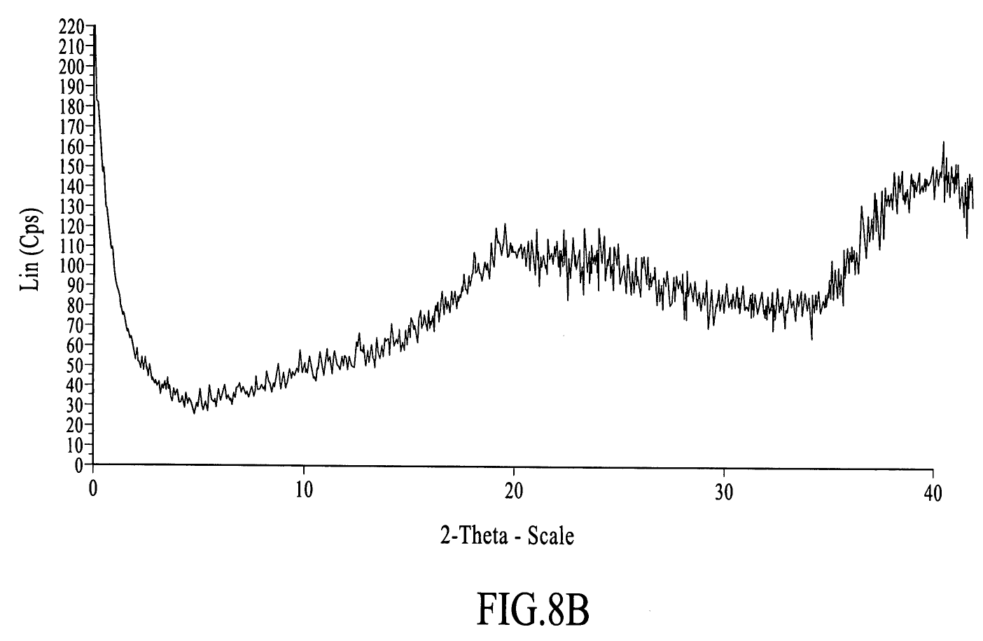

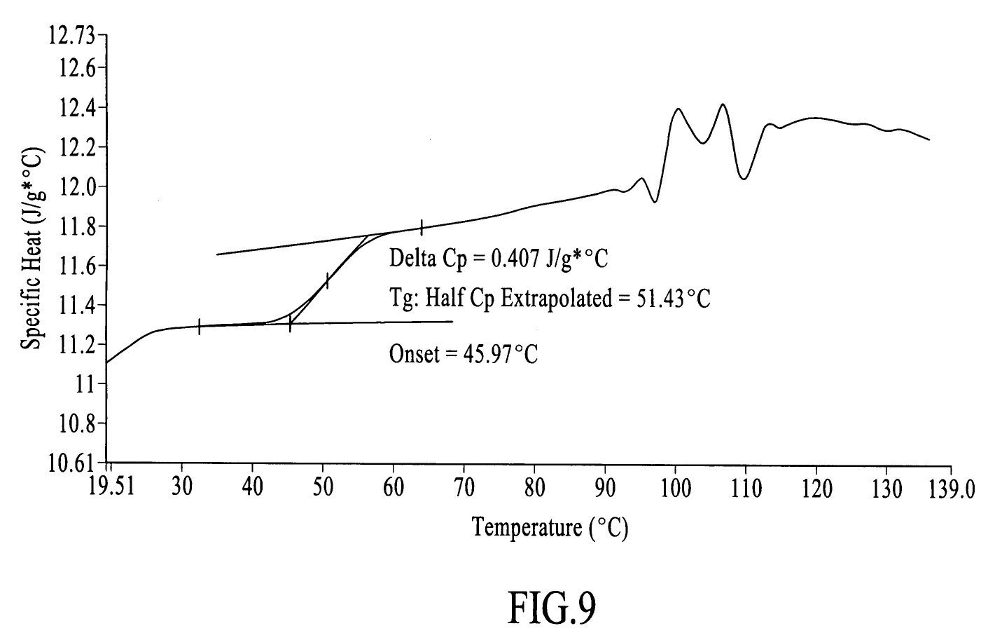

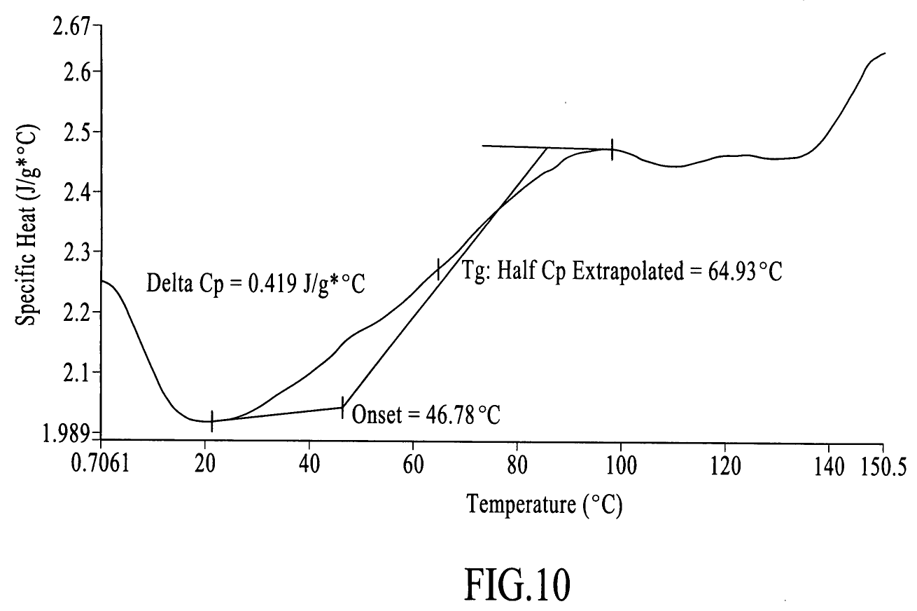

The particles comprising non-crystalline losartan potassium and stabilizing excipient made in accordance with Example 5 have been analyzed and have been found to be non-crystalline with improved physical stability. An X-ray powder diffraction pattern of the powder particles is shown in FIG. 8A. The X-ray pattern shows the powder to be non-crystalline in that no crystallinity-indicative peaks are present. The powder particles were then stored for I month at 75% relative humidity at 40° C. After this storage, the particles were X-rayed again and the X-ray powder diffraction pattern is shown in FIG. 8B. As can be seen, there is no indication of the conversion of the non-crystalline form to a crystalline form. In addition, the particles formed in accordance with Example 5 have improved glass transition temperatures over pure non-crystalline losartan particles. FIG. 9 shows a graph of the specific heat as a function of temperature for pure non-crystalline losartan particles with no excipient, produced by supercritical solvent extraction (as a SEDS™ process). FIG. 10 shows the same plot for the Example 5 particles. The particles of Example 5 have an advantageously higher T_{g} midpoint (about 65° C.) than the pure non-crystalline losartan formulation (about 51° C.) produced without excipient, but both have similar T_{g} onset values (about 46° C.).

### EXAMPLE 6

Example 6 represents another specific version of Example 4. In the production of Example 6, the following steps were carried out:

1. 4.5 g hydroxpropyl beta cyclodextrin and 0.5 g ethyl cellulose (as excipients) were added slowly to a solution containing 50 mL methanol plus 50 mL acetone. The excipients are dissolved by sonication and/or stirring at about 60 RPM.

2. 5 g crystalline losartan potassium was added to the solution made from step 1, and dissolved by sonication and/or stirring at about 60 RPM. The order of steps 1 and 2 may be reversed.

3. The solution was processed using a SED™ process using a nozzle with a 200 μm tip for the CO₂ line and a 125 μm tip for the solution line. The conditions used (at pilot plant scale) were reactor vessel pressure of 85 bar, reactor vessel temperature of 40° C., CO₂ (antisolvent) flow of 12-12.5 kg·hr⁻¹ and a solution flow of 4 ml·min⁻¹. In one or more embodiments, the result is a free flowing powder with a wet T_{g} of above about 40° C.

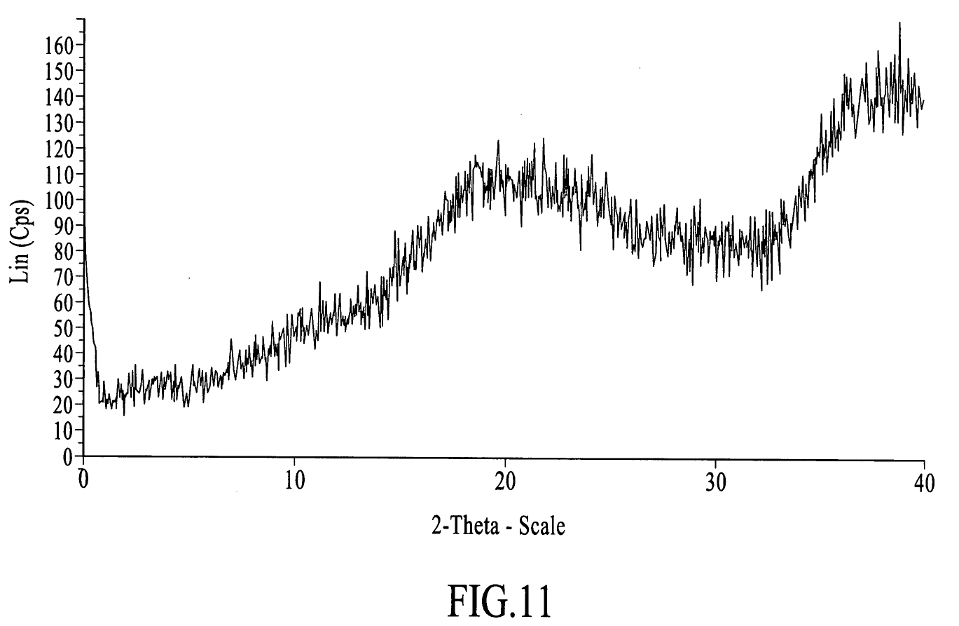

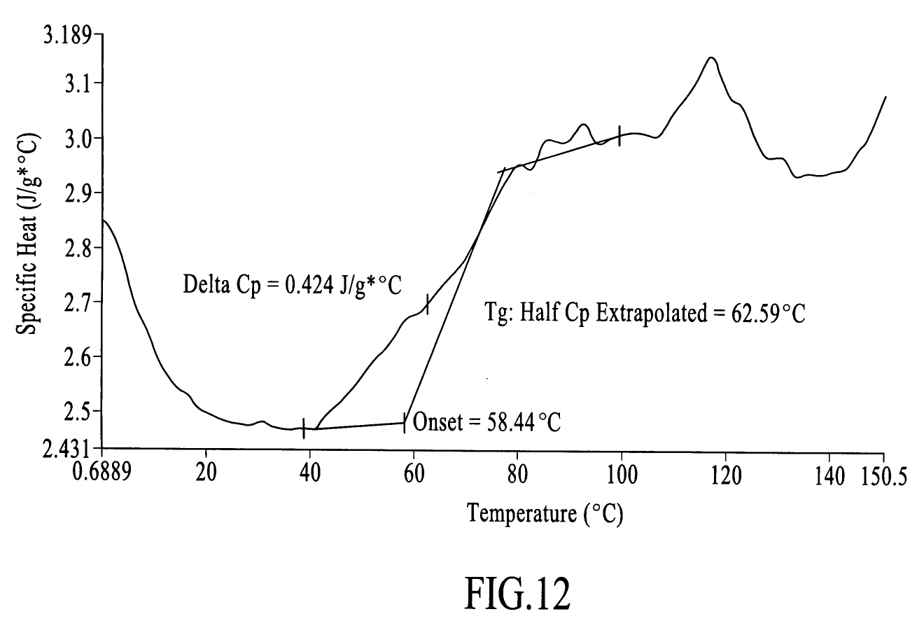

The particles comprising non-crystalline losartan potassium and stabilizing excipient made in accordance with Example 6 have been analyzed and have been found to be non-crystalline with improved physical stability. An X-ray powder diffraction pattern of the powder particles of Example 6 following storage for 2 weeks at 75% relative humidity at 40° C. is shown in FIG. 11 The X-ray pattern shows the powder to be non-crystalline in that no crystallinity-indicative peaks are present. In addition, the particles formed in accordance with Example 6 have improved glass transition temperatures over pure non-crystalline losartan particles. FIG. 12 is a graph of the specific heat as a function of temperature for the formulation of Example 6. The particles of Example 6 have an advantageously higher T_{g} midpoint (about 63° C.) than the pure non-crystalline losartan formulation (about 51° C.) produced by a SEDS™ process (compare FIG. 9).

### EXAMPLE 7

Example 7 represents another specific version of Example 4. In the production of Example 7, the following steps are carried out:

1. 3.5 g hydroxpropyl beta cyclodextrin and 1.5 g ethyl cellulose (as excipients) are added slowly to a solution containing 50 mL methanol +50 mL acetone. The excipients are dissolved by sonication and or stirring at about 60 RPM.

2. 5 g crystalline losartan potassium is added to the solution made from step 1, and dissolved by sonication and/or stirring at about 60 RPM. Note that order of step 1 and 2 may be reversed.

3. The solution is processed using a SED™ process using a nozzle with a 200 μm tip for the CO₂ line and a 125 μm tip for the solution line. The conditions used (at pilot plant scale) were reactor vessel pressure of 85 bar, reactor vessel temperature of 40° C., CO₂ (anti-solvent) flow of about 12-12.5 kg·hr⁻¹ and a solution flow of about 4 ml·min⁻¹. In one or more embodiments, the result is a free flowing powder with a wet T_{g} of above about 40° C.

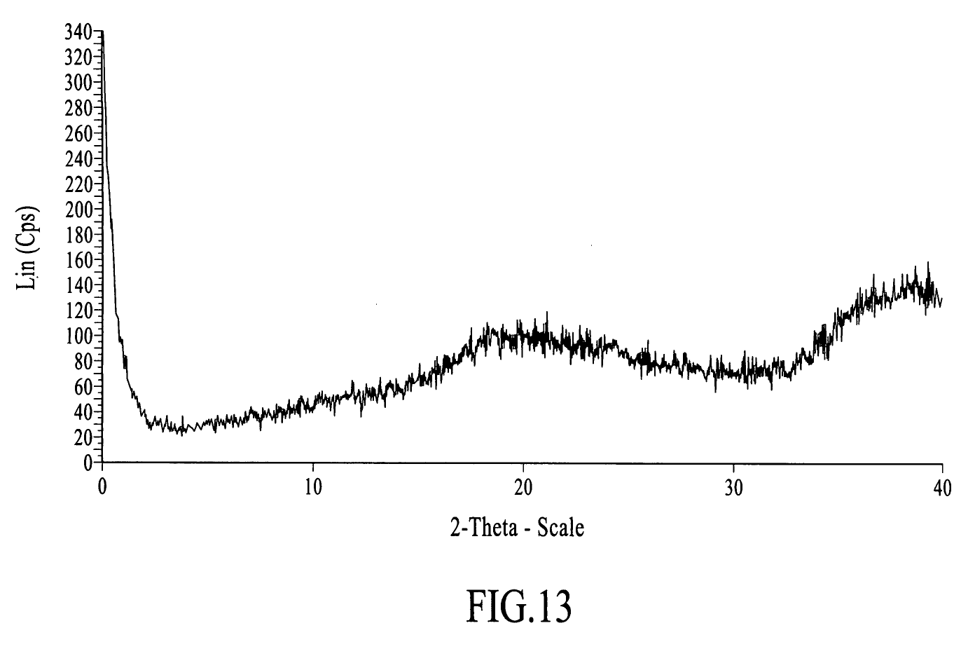

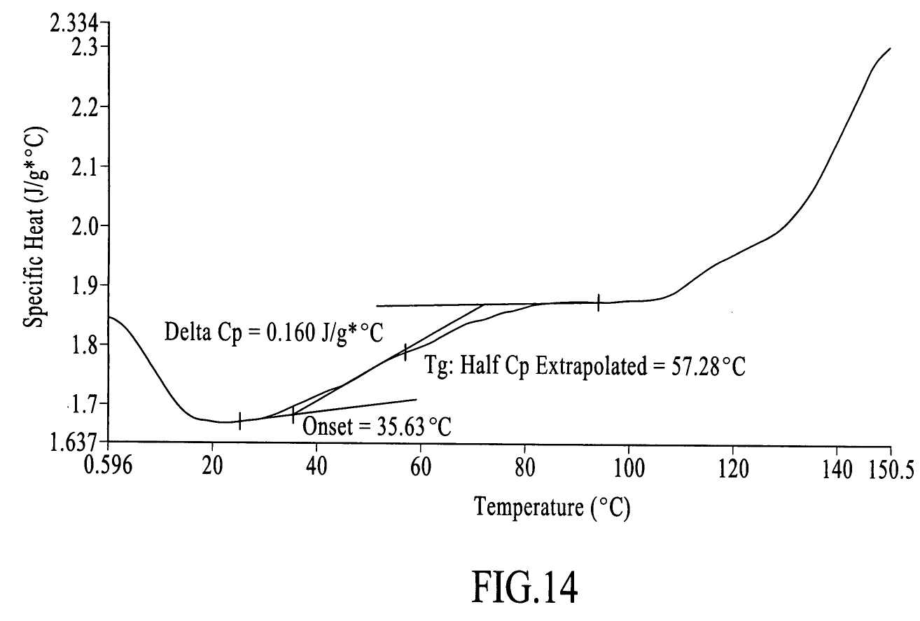

The particles comprising non-crystalline losartan potassium and stabilizing excipient made in accordance with Example 7 have been analyzed and have been found to be non-crystalline with improved physical stability. An X-ray powder diffraction pattern of the powder particles of Example 7 following storage for 2 weeks at 75% relative humidity at 40° C. is shown in FIG. 13. The X-ray pattern shows the powder to be non-crystalline in that no crystallinity-indicative peaks are present. In addition, the particles formed in accordance with Example 7 have improved glass transition temperatures over pure non-crystalline losartan particles. FIG. 14 shows a graph of the specific heat as a function of temperature for the formulation of Example 7. The particles of Example 7 have a higher T_{g} midpoint (about 57° C.) than a pure non-crystalline losartan formulation (about 51° C.) produced by SEDS™ process (compare FIG. 9).

### EXAMPLE 8

Example 8 represents another specific version of Example 4. In the production of Example 8, the following steps are carried out:

1. 5% w/v crystalline losartan potassium and 5% w/v ethyl cellulose (4 cps) are added slowly to a solution containing acetone:ethanol (85:15 v/v). The components are dissolved by sonication and/or stirring at about 60 RPM.

2. The solution is processed using a SED™ process using a nozzle with a 200 μm tip for the CO₂ line and a 125 μm tip for the solution line. The conditions used (at pilot plant scale) were reactor vessel pressure of about 85 bar, reactor vessel temperature of about 40° C., CO₂ (anti-solvent) flow of about 12-12.5 kg·hr⁻¹ and a solution flow of about 6 ml·min⁻¹. In one or more embodiments, the result is a free flowing powder with a wet T_{g} of above about 40° C.

The following tables show the resulting stability data related to the particles produced in Example 8. DSC is Differential Scanning Calorimetry, XRPD is X-ray Particle Diffraction and TGA is Thermal Gravimetric Analysis (all infra).

TABLE 1
Stability Conditions: 25° C./60% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 2.3 |
| T = 1 Week | Amorphous | Amorphous | 3.7 |
| T = 2 Weeks | Amorphous | Amorphous | 5.1 |
| T = 3 Weeks | Amorphous | Amorphous | 4.8 |
| T = 4 Weeks | Amorphous | Amorphous | 4.4 |
| T = 2 Month | Amorphous | Amorphous | 5.7 |
| T = 3 Month | Amorphous | Amorphous | 5.9 |

TABLE 2
Stability Conditions: 40° C./75% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 2.3 |
| T = 1 Week | Amorphous | Amorphous | 2.9 |
| T = 2 Weeks | Amorphous | Amorphous | 2.4 |
| T = 3 Weeks | Amorphous | Amorphous | 7.4 |
| T = 4 Weeks | Amorphous | Amorphous | 9.7 |
| T = 2 Month | Amorphous | Amorphous | 9.8 |
| T = 3 Month | Amorphous | Amorphous | 8.6 |

TABLE 3
Stability Conditions: 25° C./60% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 2.3 |
| T = 1 Week | Amorphous | Amorphous | 4.1 |
| T = 2 Weeks | Amorphous | Amorphous | 6.1 |
| T = 3 Weeks | Amorphous | Amorphous | 5.6 |
| T = 4 Weeks | Amorphous | Amorphous | 6.7 |
| T = 2 Month | Amorphous | Amorphous | 6.2 |
| T = 3 Month | Amorphous | Amorphous | 7.0 |

TABLE 4
Stability Conditions: 40° C./75% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 2.3 |
| T = 1 Week | Amorphous | Amorphous | 3.2 |
| T = 2 Weeks | Amorphous | Amorphous | 2.4 |
| T = 3 Weeks | Amorphous | Amorphous | 2.8 |
| T = 4 Weeks | Amorphous | Amorphous | 4.7 |
| T = 2 Month | Amorphous | Amorphous | 8.8 |
| T = 3 Month | Amorphous | Amorphous | 9.3 |

### EXAMPLE 9

To any of the above examples, hydrochlorothiazide may be added to the solution to allow for the production of particles comprising non-crystalline losartan, hydrochlorothiazide, and optionally a stabilizing excipient. The relative weight proportion of losartan to hydrochlorothiazide may be from 20:1 to 0.5:1, more preferably from 10:1 to 1:1, and most preferably about 4:1.

### EXAMPLE 10

Example 10 is a specific version of Example 9. In the production of Example 10, the following steps were carried out:

1. 5% w/v crystalline losartan potassium; hydrochlorothiazide and 5% w/v ethyl cellulose (4 cps) were added slowly to a solution containing acetone:ethanol (9:1 v/v) The components were dissolved by sonication and/or stirring at about 60 RPM.

2. The solution was processed using a SED™ process using a nozzle with a 200 μm tip for the CO₂ line and a 125 μm tip for the solution line.

The conditions used (at pilot plant scale) were reactor vessel pressure of about 85 bar, reactor vessel temperature of about 40° C., CO₂ (anti-solvent) flow of about 12-12.5 kg·hr⁻¹ and a solution flow of about 8 ml·min⁻¹. In one or more embodiments, the result is a free flowing powder with a wet T_{g} of above about 40° C.

The following tables 5-8 show the stability data for the particles produced in Example 10. Stability was assessed after accelerated storage, as shown in the tables, by DSC, XRPD and TGA.

TABLE 5
Stability Conditions: 25° C./60% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 1.7 |
| T = 1 Week | Amorphous | Amorphous | 3.3 |
| T = 2 Weeks | Amorphous | Amorphous | 3.1 |
| T = 3 Weeks | Amorphous | Amorphous | 3.4 |
| T = 4 Weeks | Amorphous | Amorphous | 4.2 |
| T = 2 Month | Amorphous | Amorphous | 4.7 |
| T = 3 Month | Amorphous | Amorphous | 5.2 |

TABLE 6
Stability Conditions: 40° C./75% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 1.7 |
| T = 1 Week | Amorphous | Amorphous | 3.0 |
| T = 2 Weeks | Amorphous | Amorphous | 5.1 |
| T = 3 Weeks | Amorphous | Amorphous | 5.8 |
| T = 4 Weeks | Amorphous | Amorphous | 6.0 |
| T = 2 Month | Amorphous | Amorphous | 7.0 |
| T = 3 Month | Amorphous | Amorphous | 8.1 |

TABLE 7
Stability Conditions: 25° C./60% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 1.7 |
| T = 1 Week | Amorphous | Amorphous | 3.7 |
| T = 2 Weeks | Amorphous | Amorphous | 3.6 |
| T = 3 Weeks | Amorphous | Amorphous | 4.4 |
| T = 4 Weeks | Amorphous | Amorphous | 4.1 |
| T = 2 Month | Amorphous | Amorphous | 4.2 |
| T = 3 Month | Amorphous | Amorphous | 5.0 |

TABLE 8
Stability Conditions: 40° C./75% RH Capped

| | | XRPD | TGA |
|---|---|---|---|
| | DSC | 2° and 40° 2θ in a | % weight Loss |
| | 25 to 290° C. at | stepwise mode at | 25 to 290° C. at |
| Time Point | 10° C. · min | 0.040° · 2s | 10° C. · min |
| T = 0 | Amorphous | Amorphous | 1.7 |
| T = 1 Week | Amorphous | Amorphous | 2.5 |
| T = 2 Weeks | Amorphous | Amorphous | 6.3 |
| T = 3 Weeks | Amorphous | Amorphous | 6.0 |
| T = 4 Weeks | Amorphous | Amorphous | 5.8 |
| T = 2 Month | Amorphous | Amorphous | 6.9 |
| T = 3 Month | Amorphous | Amorphous | 5.7 |

### EXAMPLE 11

In any of the above examples, the free acid of losartan may be used as the starting material instead of the crystalline losartan potassium. The free acid may be obtained as such from a commercial source, or as an intermediate in a synthetic process, or may be produced from losartan potassium, as known to the art. Thus the first step may comprise the following:

To form an aqueous solution, starting with the free acid of losartan (the free acid is not highly soluble in water), to a slurry of the losartan free acid an equal mole of KOH in water solution is added, at a final solid concentration of 0.1 to 20%, and preferably at 5-10%. To form a non-aqueous solution (e.g. organic solvent), the free acid of losartan (note that the free acid is only sparingly soluble in MeOH) is added to MeOH to form a slurry of losartan free acid and MeOH. To this slurry, a one molar equivalent of KOH or KOMe (as methanolic solution) is added, at a final solid concentration of 0.1 to 50%, and preferably at 10-20%.

In the other examples, similar substitution of free acid of losartan and alkali earth metal or alkaline earth metal and counter ion, in aqueous or organic solvent, for the crystalline losartan potassium can be made. Any of the solvent removal process disclosed herein, such as spray-drying or supercritical processing, may then may be used to produce the particles having the desired characteristics.

### EXAMPLE 12

Example 12 represents a specific version of Example 11.

1. To 200 g DI water 1.2g of KOH was added to make a basic solution. 10 g of the free acid of losartan was then added into the basic solution, and stirred until dissolved. The pH, was monitored and adjusted as necessary to a pH in the 9-10 range using IN KOH solution.

2. 11 g PVPVA was added into the solution and stirred until dissolved.

3. The solution was then spray dried with the following process conditions on a Buchi dryer: inlet temperature was about 10° C., outlet temperature was about 65° C., and a solution feed rate was about 5 mL/min. Twenty grams of powder was collected.

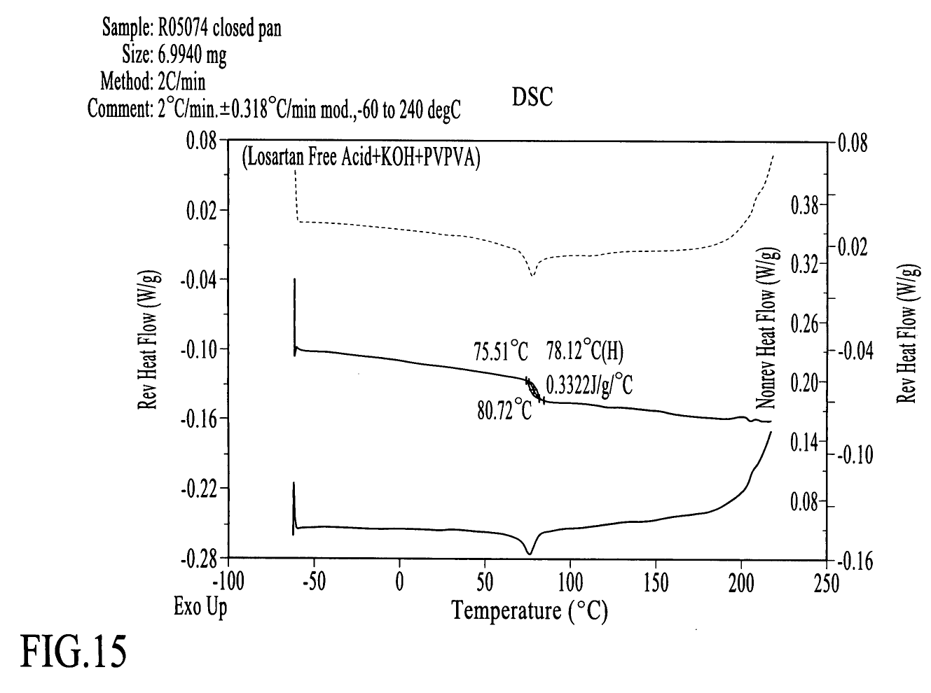

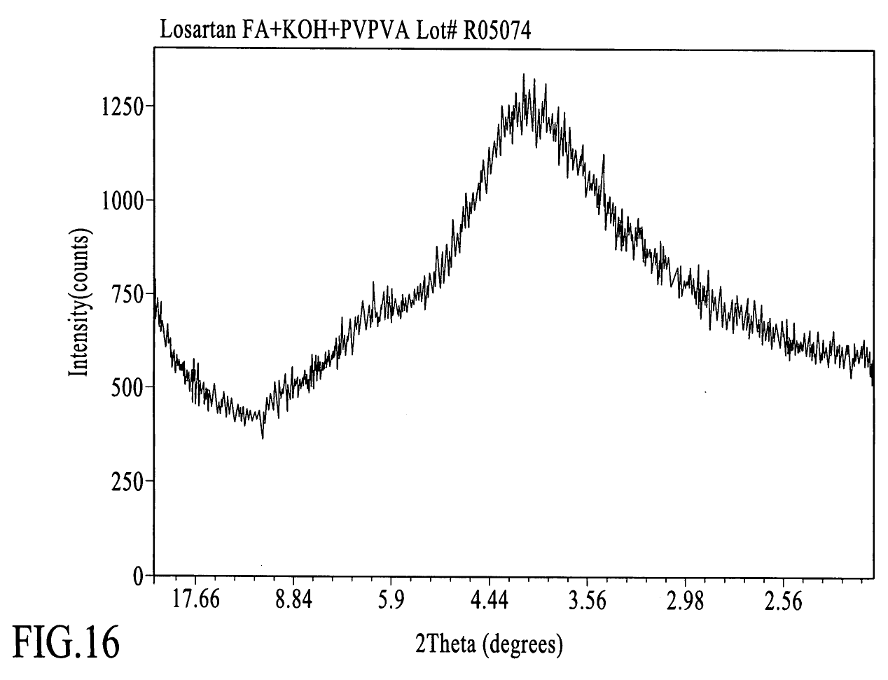

4. Differential Scanning Calorimetry (FIG. 15) and X-ray analysis results (FIG. 16) confirm that the powder made from the free acid of losartan and the PVPVA copolymer excipient is sufficiently stable, and in all respects similar to that made from losartan potassium salt plus the PVPVA copolymer. Additionally the DSC thermogram showed an advantageous T_{g} of 78° C.

### EXAMPLE 13

This Example illustrates a method of the present invention for producing non-crystalline losartan potassium, and a pharmaceutical composition of the present invention, comprising the non-crystalline losartan potassium.

The following steps were carried out: an aqueous solution of losartan potassium and polyvinylpyrrolidone vinyl acetate copolymer, at a VP:VA ratio of 60:40 was made by dissolving 2 kg of losartan potassium and 2 kg PVPVA in water. The ratio of losartan potassium:polyvinylpyrrolidone vinyl acetate copolymer was 1:1 (w/w). The solution was processed into particles by spray-drying using a rotary atomizer and a Niro spray dryer. The feed solution was about 15% solids (losartan potassium and polyvinylpyrrolidone vinyl acetate) in water. The spray dryer conditions were inlet temperature of about 150° C., and outlet temperature of about 65° C.

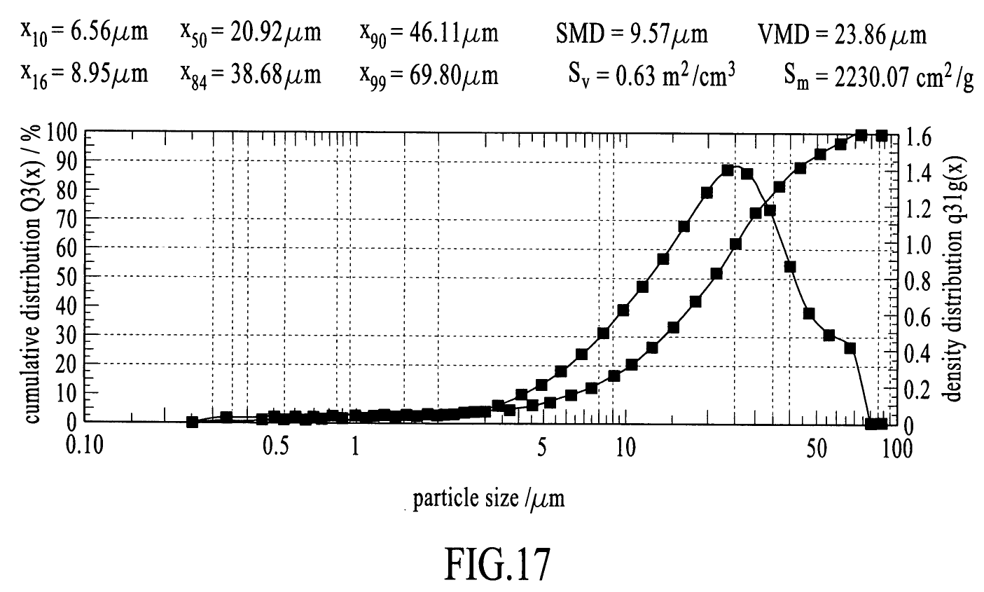

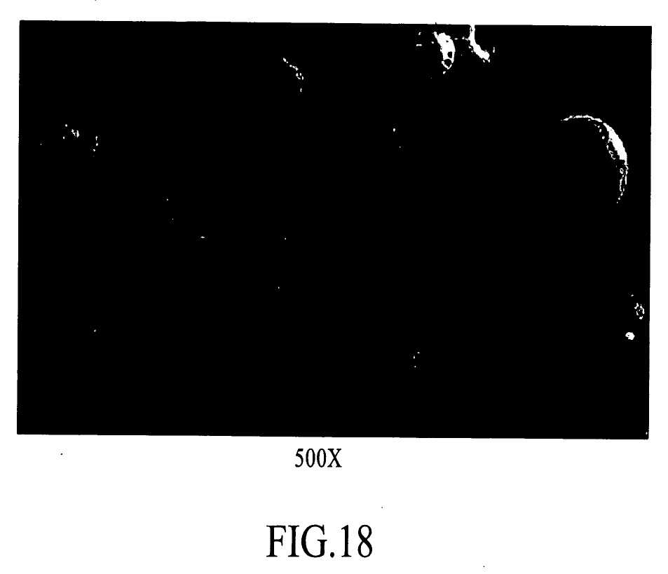

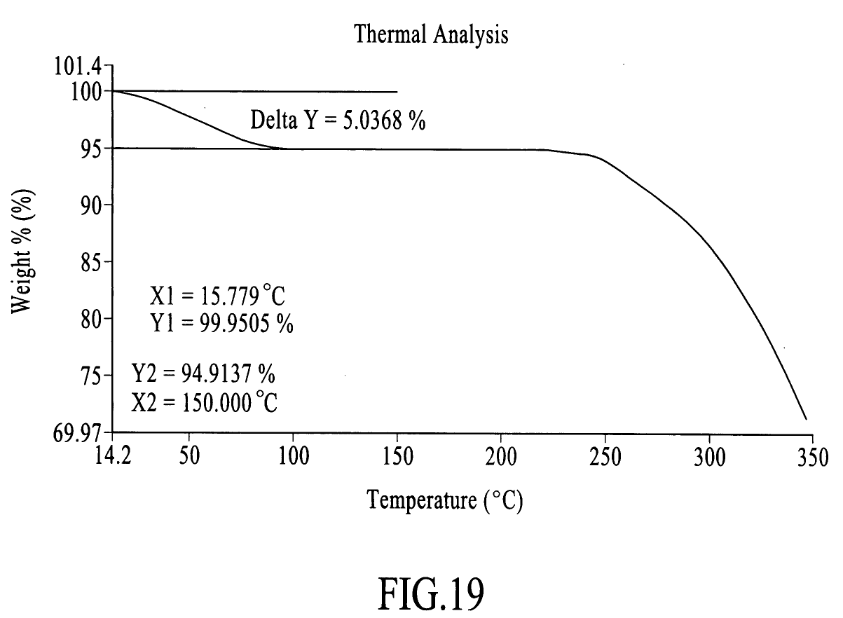

The resulting was a dry, free-flowing powder, having a particle-size distribution as shown in FIG. 17, wherein a mean particle size is 20.92 microns, 90% are below about 46.11 microns, and 10% below about 6.56 microns. The particle volume mean diameter (VMD was 23.86 microns, and SMD was 9.57 microns. FIG. 18 is a SEM micrograph, taken at a magnification of 500×, of the bulk powder showing a favorable spherical morphology. FIG. 19 is a thermal gravimetric analysis of the bulk powder, showing a water loss of about 5%.

Chemical stability was assessed after the bulk spray dried powder was packaged in HDPE containers and sealed in aluminum pouches containing silica gel desiccant. The powder was stored under conditions of 40° C./75% RH. Samples were pulled at 2, 4 and 8 weeks, and stability of the powder was assessed by HPLC. The data is presented in Table 9.

TABLE 9

| | Initial | | 2 week-40° C./75% RH | | 4 week-40° C./75% RH | | 8 week-40° C./75% RH | |
|---|---|---|---|---|---|---|---|---|
| | | Related | | Related | | Related | | Related |
| Batch No. | Assay % | Substances | Assay % | Substances | Assay % | Substances | Assay % | Substances |
| LS-003 | 99.8 | tr | 101.6 | tr | 99.2 | tr | 101.9 | Nil |
| LS-004 | 99.6 | tr | 99.6 | tr | 98.7 | tr | 98.5 | Nil |

tr-Traces below LOQ {0.022%}

A spray-dried bulk powder formulation as described in Example 13 was prepared as a pharmaceutical composition, comprising a tablet for oral dosage. The composition is given in Table 10.

Bulk spray dried powder of 50% losartan potassium:PVPVA powder, lactose monohydrate and microcrystalline cellulose are blended along with magnesium stearate and compacted using a roller compactor. The compacts are sieved, blended with magnesium stearate and compressed into tablets. The formulation of this Table includes no binder or disintegrant as such.

TABLE 10

| Ingredients | mg/tab |
|---|---|
| Spray dried 50 w/w % Losartan | 200 |
| Potassium: PVPVA powder | |
| Lactose Monohydrate | 49 |
| Microcrystalline cellulose | 49 |
| Aerosil | 2 |
| Magnesium stearate | 5 |
| Coat (3.28% w/w)-Opadry White | 10 |
| Total | 315 |

Tablets prepared as above were packaged in HDPE containers, which in turn were sealed in aluminum poches containing silica gel desiccant. The tablets are assessed for physical dimensions, dissolution initially, and after storage for up to three months under conditions of 25° C./60% RH. Chemical stability was assayed by HPLC after storage for up to three months under conditions of 25° C./60% RH, and 40° C./75% RH. Results of physical dimension stability are presented in Table 11 below.

TABLE 11

| Parameters | Initial | 1 Month | 2 Months | 3 Months |
|---|---|---|---|---|
| Thickness (mm) | 4.52-4.66 | 4.58-4.77 | 4.58-4.72 | 4.58-4.71 |
| Hardness (N) | 132-151 | 118-146 | 130-146 | 116-142 |
| Disintegration | 18 min., | 11 min., | 14 min., | 15 min., |
| Time (min., sec.) | 5 secs. | 3 sec. | 19 sec. | 3 sec. |

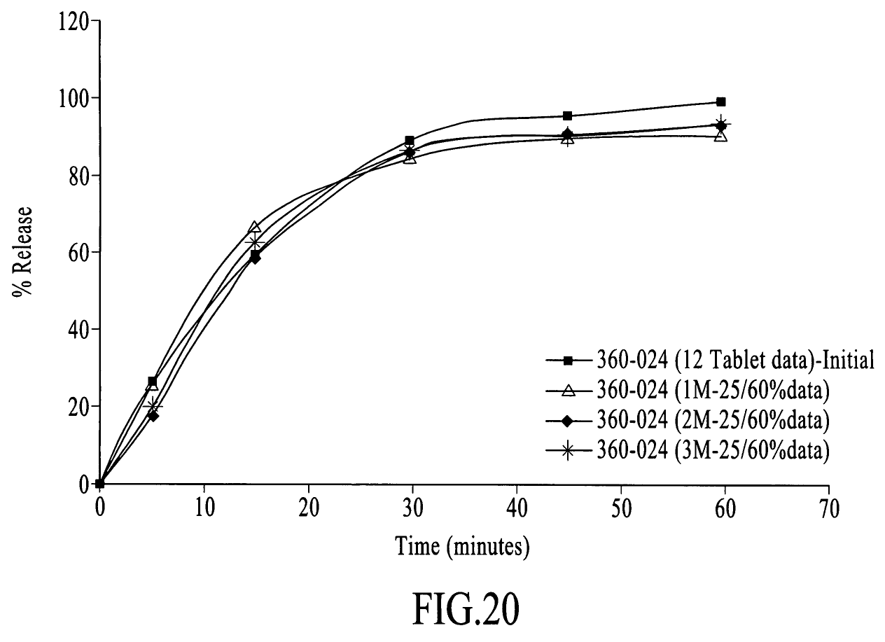

Dissolution testing. The dissolution of the tablet dosage formulation was measured in USP II dissolution apparatus in water at 50 RPM. The samples were analyzed with a UV spectrophotometer at a wavelength of 256 nm. Table 12 below presents dissolution data of the tablets of Example 13, compared to the COZAAR® 100 mg tablet. As can be seen from the Table 12, the dissolution profile of the inventive tablets closely matches that of the commercially-available tablets. Additionally, the dissolution profiles of the inventive tablets of Example 13 over a three month period at 25° C./60% RH are shown in FIG. 20. The profiles show that dissolution stability is good, with no significant degradation of the dissolution profile over the test period. The tablet formulation is physically and chemically stable for at least about six months, preferably stable for at least about one year, and more preferably stable for at least about two years, all under ambient conditions.

TABLE 12

| | | Percent Release | | |
|---|---|---|---|---|
| | | (average of 12 | Standard | RMS |
| | Time (minutes) | tablets) | Deviation | Deviation |
| COZAAR ® | 30 | 84.1 | 15.3 | 18.2 |
| tablets | 60 | 97.5 | 9.9 | 10.2 |
| Present | 30 | 90.0 | 9.0 | 10.0 |
| Invention | 60 | 99.2 | 5.9 | 6.0 |

Chemical stability of the tablet dosage formulation was assayed by HPLC. Results are shown in Table 13 below. It can be seen by comparing the percentage of active at To with that after 1, 2 and 3 months of accelerated storage, that there was essentially no change in active percentage, nor were there any significant levels of related substances detected.

TABLE 13

| | Related Substances | | | | | | | | | | |
|---|---|---|---|---|---|---|---|---|---|---|---|
| | | | | | RRT | RRT | RRT | | RRT | Total | |
| Time Point | RRT 0.44 | RRT 0.55 | RRT0.64 | RRT0.83 | 0.88-0.90 | 1.3-1.4 | 1.6-1.76 | RRT1.8 | 3.0-3.2 | Impurity | Assay in % |
| Initial | — | tr | tr | tr | tr | — | — | — | tr | tr | 93.2 |
| 25° C./60% RH 1M | tr | tr | tr | tr | tr | — | — | — | tr | — | 93.2 |
| 40° C./75% RH 1M | tr | tr | tr | tr | tr | — | tr | — | 0.03 | 0.03 | 93.4 |
| 25° C./60% RH 3M | — | — | tr | — | tr | — | — | tr | 0.04 | 0.04 | 92.4 |
| 25° C./60% RH 2M | — | tr | tr | — | tr | — | — | — | 0.04 | 0.04 | 92.5 |
| 40° C./75% RH 2M | — | — | tr | — | tr | — | — | — | 0.07 | 0.07 | 92.2 |
| 40° C./75% RH 3M | — | tr | tr | — | tr | tr | tr | — | 0.08 | 0.08 | 92.1 |

tr-Traces below LOQ {0.022%}
RRT-Relative Retention Time

The above test results show that the amorphous bulk spray dried losartan potassium:PVPVA (1:1) powder is physically and chemically stable.

The tablet dosage formulated from the bulk amorphous powder has likewise been shown to be physically and chemically stable. As shown in Table 12, the dissolution profile of this tablet dosage form closely matches that of the commercially-available COZAAR® 100 mg tablet.

### EXAMPLE 14

This Example illustrates a method (roller compaction process) by which a tablet dosage formulation may be made in accordance with one or more embodiments of the present invention.

1. Sift losartan potassium:PVPVA (1:1) made in accordance with one or more process embodiment herein and Microcystalline cellulose (as Avicel pH 102) through 40 mesh sieve and collect in a stainless steel container.

2. Lactose DCL 15 and Aerosil (a silicone dioxide), is mixed and sifted through a 40 mesh sieve, and collect in a stainless steel container.

3. The sifted ingredients of steps 1 and 2 are charged into a drum blender and mixed for 30 minutes at speed of 22 rpm.

4. Sift magnesium stearate through a 60-mesh sieve and add to the above blend in the drum blender and mix for 5 minutes at 22 rpm.

5. Roll compact the above mass using corrugated rollers at a compaction pressure of 3-7 tons.

6. Size the compacts through 18 mesh sieve using an oscillating granulator.

7 Sift magnesium stearate through 60mesh sieve; add to sized granules of step 6 in the drum blender and mix for 5 minutes at about 22 rpm.

Compress the above blend using 14×7 mm oval shaped standard concave punches with upper punch embossed with 40 and lower punch plain.

Coat the tablets using Opadry white dispersion in 80% IPA.

### EXAMPLE 15

Any of the above examples may be administered to a patent (human or animal), for a condition treatable thereby, and particularly to treat a patient having hypertension and/or congestive heart failure. For example, the formulations described herein may be formulated into a tablet containing 25 mg, 50 mg, or 100 mg of losartan potassium. Alternatively, the formulations may be formulated into a tablet containing 50 mg of losartan potassium with 12.5 mg of hydrochlorothiazide or 100 mg of losartan potassium with 25 mg of hydrochlorothiazide. These amounts may be altered in order to achieve a desired therapeutic profile.

### EXAMPLE 16

Example 16 is a specific version of Example 15, illustrating pharmacokinetic performance of the tablet dosage form, as formulated in Example 13, in human subjects.

A spray dried Losartan potassium:PVPVA (1:1) powder formulated as a tablet in accordance with one or more embodiments herein was tested against a commercially available COZAAR® tablet in a crossover comparative pharmacokinetics trial. Healthy human adult male subjects were administered the losartan tablet (100 mg) of the present invention and the COZAAR® tablet (100 mg).

After a supervised overnight fast, subjects received a single oral dose of the assigned formulation, with 240 mL of water. At least 7 days between doses were required for washout. Twelve subjects completed all 4 arms and 1 subject completed all legs except the reference formulation arm. The twelve subjects completing all 4 arms were used in the pharmacokinetic assessment. Blood samples were collected pre-dose and at 0.167 (10 min), 0.333 (20 min), 0.5 (30 min), 0.667 (40 min), 0.833 (50 min), 1, 1.25, 1.5, 1.75, 2, 3, 4, 5, 6, 7, 8, 10, 12, 18, 24, 30, 36 and 48 hr post dose to define the plasma parent (losartan) and metabolite (5-carboxylic acid) concentration-time profiles.

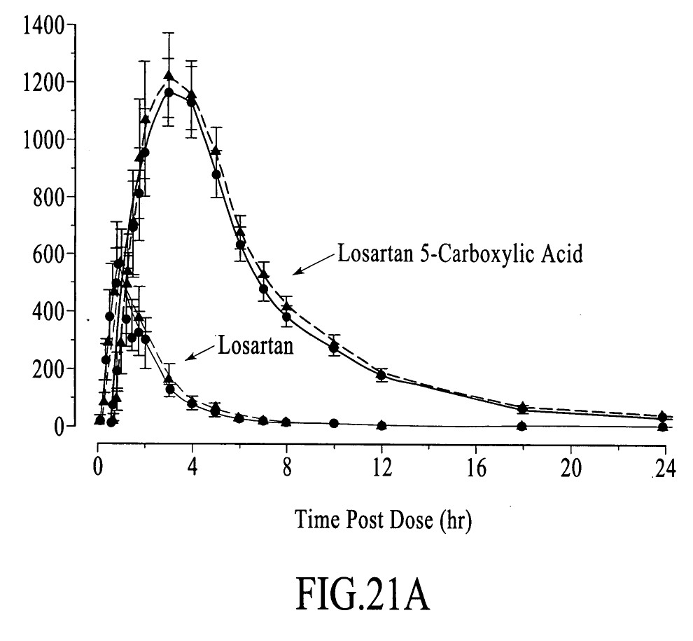

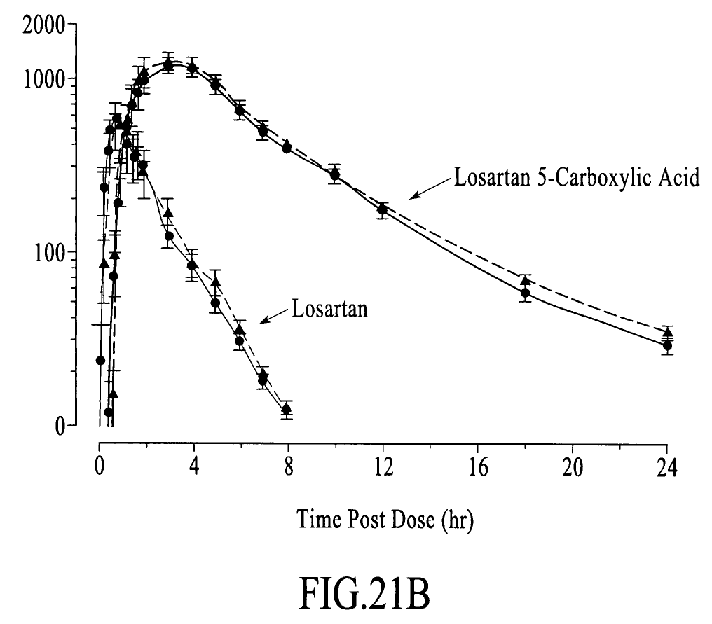

The mean plasma parent and metabolite concentration-time profiles for the tablet comprising non-crystalline losartan potassium and PVPVA in a 1:1 ratio, made in accordance with one or more embodiments herein are shown in FIGS. 21A and 21B and are compared to those of a commercially-available losartan COZAAR® tablet. In the Figures, the curves labeled with triangle-symbols/dashed-lines represent the COZAAR® reference formulation, while the curves labeled with the circle-symbols/solid-lines represent the formulation of the present invention. The profiles for losartan parent (lighter colors) and 5-carboxylic acid metabolite (darker colors) are also annotated separately. It can be seen that the concentration-time profiles closely match between formulations. The pharmacokinetics comparison/bioequivalence assessment results of this pilot trial are presented in Table 14 below.

TABLE 14

| | | Losartan | 5-Carboxylic Acid |
|---|---|---|---|
| Cmax | Arithmetic Mean (% CV) | 792.845 | 1392.729 |
| | Nektar Formulation “A” | (51.09) | (30.10) |
| | Arithmetic Mean (% CV) | 917.251 | 1475.213 |
| | Reference Formulation “D” | (57.18) | (31.86) |
| | Ratio (% Ref) | 89.44 | 94.55 |
| | 90% Confidence Interval | 58.28-137.27* | 78.44-113.97* |
| AUC | Arithmetic Mean (% CV) | 1138.473 | 8143.148 |
| (0-t) | Nektar Formulation “A” | (30.53) | (26.67) |
| | Arithmetic Mean (% CV) | 1212.356 | 8379.736 |
| | Reference Formulation “D” | (26.90) | (27.01) |
| | Ratio (% Ref) | 93.27 | 97.23 |
| | 90% Confidence Interval | 78.41-110.94* | 86.33-109.51 |
| AUC | Arithmetic Mean (% CV) | 1155.397 | 8195.147 |
| (0-∞) | Nektar Formulation “A” | (30.16) | (26.51) |
| | Arithmetic Mean (%CV) | 1227.478 | 8437.053 |
| | Reference Formulation “D” | (26.80) | (26.94) |
| | Ratio (% Ref) | 93.55 | 97.21 |
| | 90% Confidence Interval | 78.97-110.82* | 86.39-109.39 |

Bioequivalence of the two formulations is established where the 90% confidence intervals of the relative mean log-transformed Cmax and AUC of the test (Nektar Formulation) to reference (Cozaar Reference Formulation) formulation fall within 80-125%. For losartan potassium, both the parent (losartan) and active metabolite (losartan 5-carboxylic acid) should meet these bioequivalence requirements.

The data show that the observed pharmacokinetic parameters are on target for bioequivalence, but the 90% confidence intervals are wider than desired (outside 80-125%) for bioequivalence. This observation is due to the small sample size used in this pilot trial (sample size n=12 subjects for reference formulation and n=12 subjects for Nektar's formulation). Bioequivalency requirements are likely to be achieved if the two formulations are compared in a trial of sufficient sample size.

### EXAMPLE 17

An example according to the present invention involves the formation of pure non-crystalline losartan by a Solution Enhanced Dispersion by Supercritical fluids (SEDS™) process, such as the one described in U.S. Pat. No. 5,851,453 and U.S. Pat. No. 6,063,138, both of which are incorporated herein by reference in their entireties, and with particular regard to supercritical process methods, steps, materials, and conditions. Losartan is dissolved in an organic solution, such as an organic solution comprising methanol and optionally acetone. The solution is then contacted by supercritical carbon dioxide which extracts the losartan to produce particles comprising losartan. The starting material may be one or more of the crystalline polymorphs of losartan potassium. The crystalline losartan potassium is sufficiently soluble in organic solvents. The process is performed under conditions selected to result in the formation of a non-crystalline form of losartan.

Specifically, the non-crystalline losartan potassium of Example 17 can be made by performing the following steps:

1. Starting with the commercially available crystalline losartan potassium, the salt is dissolved in an organic solvent comprising MeOH at 1-20%, preferably at 2.5-10% solids content.

2. The solution is then contacted, in a particle precipitation process, with a supercritical or near critical fluid anti-solvent, such as supercritical CO₂, which extracts the which extracts the losartan potassium from the solution.

The solution of step 1 can alternatively or additionally be made into powder using technologies known in the field, such as by vacuum drying, bubble drying, freeze drying, spray-freeze drying, evaporation, or extraction. This process can be performed in other organic solvents. For example, useful solvents comprise ethanol, iso-propanol, methanol, other short chain alcohols, esters, ethers, and other low boiling point solvents.

Analytical Methods

The analytical techniques employed in some of the examples are more fully described below.

X-Ray Powder Diffraction (XRD/XRPD)

XRD/XRPD was used to characterize the nature of a sample or samples. An amorphous sample is indicated by the lack of diffraction peaks in the diffraction pattern which is characteristic of crystalline materials. Samples were analysed (on a D5000 XRD (Siemens, Germany) between 2 and 400 2θ, at a scan rate of 0.02 degrees per second, unless indicated otherwise.

Scanning Electron Microscopy (SEM)

Particle size and morphology were investigated using a FEI XL30 TMP Scanning Electron Microscope. SEM was used to observe the morphology of the particles before and after exposure to moisture. Samples were mounted on silicon wafers that were then mounted on top of double-sided carbon tape on an aluminum SEM stub. The mounted powders were then sputter-coated with gold: palladium in a Denton sputter-coater for 60 to 90 seconds at 75 mTorr and 42 mA. This produces a coating thickness of approximately 150 Å. Images were taken with a Philips XL30 ESEM operated in high vacuum mode using an Everhart-Thomley detector to capture secondary electrons for the image composition. The accelerating voltage was set at 20 kV using a LaB6 source. The working distance was between 5 and 6 mm.

Differential Scanning Calorimetry (DSC)

DSC was used to determine glass transition temperatures. This technique provides a measure of the glass transition characteristics of amorphous materials. In addition, the absence of a melting point is indicative of the lack of three dimensional order characteristic of crystalline materials. A Perkin-Elmer™ DSC 7 (Perkin-Elmer Ltd, UK) was used. 1-5 mg samples were examined in sealed, crimped aluminium pans, under an atmosphere of nitrogen. Samples were measured using a TA DSC-2920 instrument (TA Instruments, New Castle, Del.). About 5-10 mg sample was packed into an aluminum DSC pan and gently tapped to get the powder to form a uniform layer on the bottom of the pan (Catalog numbers 900 793.901 for pans and 900 794.901 for lids). The DSC pan was hermetically sealed using a sample encapsulation press (part # 900680.902). Helium is used as the DSC purge gas at 30 ml/min. A Refrigerated Control System (RCS) provides the heat sink for the DSC, with helium as the circuit gas run at ˜110 ml/min. In modulated DSC experiments, the sample was first cooled to about 0° C., held isothermally for 10 minutes, and then heated at 2° C./minute to ˜200° C. The heating rate was modulated by superimposing a sinusoidal heating profile at ±0.318° C./min.

Thermogravimetric Analysis (TGA)

This method was used to assess changes in water content of the product during storage by measuring the loss of mass on heating. The sample weight loss at elevated temperatures was measured using TGA-2950 instrument made by TA Instruments. The sample was immediately heated, in order to minimize the initial dehydration by the dry nitrogen gas, from room temperature to 250° C. at a rate of 2° C./min and/or 0.2° C./min. The % weight loss was calculated using the TA software.

Dynamic Vapor Sorption

The moisture sorption isotherm of a powder at 25° C. was measured using a dynamic vapor sorption (DVS) instrument made by Surface Measurement Systems, UK. Sample masses between 5 and 20 mg were used. Samples were loaded in a dry box to avoid moisture sorption. In the first step of the experimental run, the sample was dried at 25° C. and 0% RH for at least 300 minutes, in an attempt to bring the sample to near zero wt % water. Then, the instrument was programmed to increase the RH in steps of 5% RH from 0% to 90% RH and decrease the RH in steps of 5%RH from 90% to 0% RH. A criterion of dm/dt=0.0001%/min was chosen for the system to hold at each RH step before proceeding to the next RH step.

High Performance Liquid Chromatography

A Hypersil model BDS C18 (25 cm×4.6 mm) column was used at ambient temperature. The mobile phase contained 30:70 acetonitrile: phosphate buffer (ammonium dihydrogen phosphate), with pH adjusted to 2.3 using Ortho-phosphoric acid. The flow rate was 1 ml/min and effluent was monitored at 254 nm, in isocratic mode. Injection volume was 50 μl, with losartan target concentration about 40 μg/ml.

Dissolution Method

A USPII dissolution apparatus was used to measure dissolution of the formulations in de-aerated water at 50 RPM. The samples were analyzed with a UV Spectrophotometer at 256 nm.

UV Spectrophotometry—The weight fraction of drug in samples was measured with an Ultrospec™ 4000 spectrophotometer (Pharmacia Biotech, Cambridge, England), from reconstituted solutions of the samples. The absorbance of the polymers was negligible at the wavelengths used.

Although the present invention has been described in considerable detail with regard to certain preferred versions thereof, other versions are possible, and alterations, permutations and equivalents of the version shown will become apparent to those skilled in the art upon a reading of the specification. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

1. A composition comprising non-crystalline losartan potassium and a stabilizing excipient, wherein in that the composition is physically and chemically stable for at least one year at about 25° C. and about 60% RH.

2. The composition of claim 1 wherein the composition comprises a bioequivalence at least approximately equal to that of substantially crystalline losartan potassium.

3. A pharmaceutical or nutraceutical composition comprising a formulation according to claim 1.

4. The composition of claim 1 wherein the non-crystalline losartan is produced by the steps of (a) preparing a solution comprising losartan, a stabilizing excipient and a solvent;
(b) atomizing the solution comprising losartan, stabilizing and solvent; and
(c) spray-drying the losartan, stabilizing excipient and solvent solution; wherein a plurality of particles, in the form of a free-flowing powder, result.

5. The composition of claim 5 wherein the losartan comprises the potassium salt, in an amount of between about 0.1 to 25% by weight, and the stabilizing excipient comprises a vinyl pyrrolidone/vinyl acetate co-polymer.

6. The composition of claim 4 wherein the losartan solution of step (a) is produced by the steps of (i) providing a free-acid form of losartan; and
(ii) combining the free-acid form of losartan with a substantially equimolar amount of a compound comprising an alkali earth metal or alkaline earth metal and a counter ion in a solvent.

7. The composition of claim 1 wherein the non-crystalline losartan is produced by the steps of (a) preparing a solution comprising losartan, a stabilizing excipient and a solvent;
(b) atomizing the solution comprising losartan, stabilizing excipient and solvent; and
(c) removing, under supercritical or near-critical conditions, the solvent; wherein a plurality of particles, in the form of a free-flowing powder, result.

8. The composition of claim 7 wherein the stabilizing excipient comprises a vinyl pyrrolidone/vinyl acetate co-polymer.

9. The composition of claim 1 wherein the composition comprising non-crystalline losartan is produced by the steps of
(a) providing a free-acid form of losartan;
(b) combining the free-acid form of losartan with a substantially equimolar amount of a compound comprising an alkali earth metal or alkaline earth metal and a counter ion in a solvent to form an acid salt;
(c) preparing a solution comprising the losartan, a stabilizing excipient and a solvent;
(d) atomizing the solution comprising the losartan, stabilizing excipient and solvent; and
(e) removing the liquid from the solution of the losartan, stabilizing excipient and solvent, wherein a plurality of particles, in the form of a free-flowing powder, result.

10. The composition of claim 1 wherein the excipient is oligomeric or polymeric.

11. The composition of claim 10 wherein the excipient has a higher T_{g}, a lower hygroscopicity, or both, compared to the non-crystalline losartan alone.

12. The composition of claim 10 wherein the composition has a T_{g} of above about 40° C.

13. The composition of claim 10 wherein the excipient comprises polymers of vinyl acetate, HPMC, HPC, cellulose and cellulose derivatives, tris, hydroxypropyl beta cyclodextrin, and copolymers of vinyl pyrrolidone with vinyl acetate, and mixtures thereof.

14. The composition of claim 13 wherein the excipient comprises a vinyl pyrrolidone vinyl acetate co-polymer in a ratio of vinyl pyrrolidone:vinyl acetate of between about 80:20 to 20:80.

15. The composition of claim 13 wherein the excipient comprises a vinyl pyrrolidone vinyl acetate co-polymer in a ratio of vinyl pyrrolidone:vinyl acetate of about 60:40.

16. The composition of claim 13 wherein the composition comprising non- crystalline losartan and excipient is produced by atomizing and spray-drying a solution comprising losartan, excipient and solvent, and wherein a free-flowing powder results.

17. The composition of claim 13 wherein the composition comprising non- crystalline losartan and excipient is produced by a supercritical particle extraction process from a target solution/suspension comprising losartan, excipient and solvent, and wherein a free-flowing powder results.

18. The formulation according to claim 17 wherein the process further comprises contacting the target solution with a compressed fluid anti-solvent under conditions which allow the anti-solvent simultaneously both to disperse the target solution and to extract the vehicle from it so as to cause particles of losartan and excipient to precipitate as a co-formulation.

19. The formulation of claim 1 wherein the losartan comprises 2-butyl-4-chloro-1-[(2′-tetrazol-5-yl)-biphenyl-4-yl]methyl]-5-(hydroxymethyl) imidazole.

20. A method of preparing a particulate co-formulation comprising losartan and a stabilizing excipient, the method comprising providing a solution or suspension of losartan and a stabilizing excipient in a solvent; and
extracting the liquid from the solution or suspension so as to cause particles of the formulated losartan and stabilizing excipient to precipitate, wherein a plurality of particles result, the particles the form of a free-flowing powder, and having a T_{g} of about 40° C. or greater, a residual moisture of about 3-5%, and a volume mean diameter of about 5-200 microns.

21. The method of claim 20 wherein the stabilizing excipient comprises copolymer of vinyl pyrrolidone and vinyl acetate, in a ratio of vinyl pyrrolidone:vinyl acetate of about 60:40.

22. The method of claim 21 wherein the liquid is extracted by spray-drying.

23. The method of claim 21 wherein the liquid is extracted by supercritical or near-critical solvent extraction.

24. A solid, free-flowing, non-cystalline formulation comprising losartan and a stabilizing excipient, wherein the formulation when stored at 40° C. and 75% relative humidity converts to a crystalline form more slowly than a formulation without the stabilizing excipient.

25. A method of co-forming losartan and an excipient the method comprising providing a quantity of losartan and a quantity of excipient;
mixing the losartan and excipient;
heating the losartan and excipient mixture to a temperature and for a time sufficient to cause the losartan and excipient to form a homogeneous hot-melt; and
processing the homogenous hot melt into particles, whereby a plurality of particles result, the particles in the form of a free-flowing powder.

26. The method of claim 25 whereby the processing comprises spray congealing, melt extrusion or a combination thereof.

27. A solid, free-flowing, non-cystalline formulation consisting essentially of losartan, wherein the formulation is prepared by the steps of:
(a) preparing a solution comprising losartan potassium and a solvent;
(b) atomizing the solution comprising losartan and solvent; and
(c) removing, under supercritical or near-critical conditions, the solvent; wherein a plurality of particles result.

28. A tablet form of a pharmaceutical composition comprising non-crystalline losartan and a stabilizing excipient, wherein the composition is stable for at least about three months.

29. The tablet of claim 28 wherein the tablet provides a bioequivalence, on a dose per dose basis, substantially as a COZAAR® tablet.

30. The tablet of claim 28 wherein the composition exhibits a morphology substantially as shown in at least one of FIG. 18, an X-ray diffraction pattern substantially as shown in at least one of FIGS. 5, or a combination thereof.

31. The tablet of claim 28 wherein the stabilizing excipient comprises a vinyl pyrrolidone/vinyl acetate co-polymer, and is present in a weight ratio to the losartan of between about 0.1:10 to about 10:0. 1, and wherein the tablet further comprises lactose monohydrate, microcrystalline cellulose, magnesium stearate, silicon dioxide and a coating agent.

32. The tablet of claim 31 wherein the vinyl pyrrolidone/vinyl acetate co-polymer is present in a weight ratio to the losartan of about 1:1.

33. The tablet of claim 31 wherein the tablet is physically and chemically stable for at least about three months.

34. The tablet of claim 31 and further including a diuretic-effective amount of a hydrochlorothiazide.

35. The tablet of claim 28 made by a process comprising;
providing a free-flowing powder comprising non-crystalline losartan and an excipient;
granulating the powder; and
compacting the granulated powder to form a tablet.

36. A pharmaceutical composition in tablet form consisting essentially of a non- crystalline losartan, a stabilizing amount of a vinyl pyrrolidone/vinyl acetate co- polymer, and optional tablet processing agents, wherein the losartan and the polymer are present in a ratio of about 1:1, and wherein the tablet provides a bioequivalence, on a per dose basis, substantially as a COZAAR® tablet.

37. The composition of claim 36 wherein the composition is physically and chemically stable for at least about three months.

38. The composition of claim 36 wherein the optional tablet processing agents are selected from lactose monohydrates, microcrystalline celluloses, magnesium stearates, silicone dioxide, coating agents and mixtures thereof.

39. The composition of claim 36 wherein at least about 90% of the tablet is dissolved or dispersed in water within about 30 minutes.

40. The composition of claim 36 and further including a hydrochlorothiazide.

41. A process for making a tablet dosage form comprising non-crystalline losartan, the process comprising
(a) sizing a quantity of particles comprising losartan potassium and stabilizing excipient with microcystalline cellulose through a sieve;
(b) mixing lactose and silicone dioxide;
(c) blending the ingredients of steps (a) and (b);
(d) sizing a first quantity of magnesium stearate through a sieve and adding to the blend of step (c) while continuing to blend;
(e) compacting the blend of step (d);
(f) sizing the compact through a sieve;
(g) sizing a second quantity of magnesium stearate through a sieve and adding to the sized granules of step (f), with continued mixing;
(h) compressing the blend of step (g) to result in tablets; and
(i) coating the tablets with a tablet coating agent.
